(19) United States
(12) Patent Application Publication
(10) Pub. No.: US 2017/0183637 A1
(43) Pub. Date: Jun. 29, 2017

(54) SPLIT RECOMBINANT LUCIFERASE, AND ANALYSIS METHOD USING THEREOF

(71) Applicant: OLYMPUS CORPORATION, Tokyo (JP)

(72) Inventor: Takashi SUGIYAMA, Tokyo (JP)

(73) Assignee: OLYMPUS CORPORATION, Tokyo (JP)

(21) Appl. No.: 15/451,939

(22) Filed: Mar. 7, 2017

Related U.S. Application Data
(63) Continuation of application No. PCT/JP2014/074606, filed on Sep. 10, 2014.

(51) Int. Cl. C12N 9/02; C12N 15/52; C12Q 1/26; C12N 15/62

(52) U.S. Cl. CPC C12N 9/0069; C12N 15/62; C12N 15/52; C12Q 1/26

(57) ABSTRACT

Disclosed is a split recombinant protein that includes N-terminal and C-terminal fragments of a firefly luciferase, and a linker peptide. The N-terminal fragment is one of two fragments of a firefly luciferase split into two at a splitting position specific to the firefly luciferase. The C-terminal fragment includes a C-terminal fragment of a firefly luciferase split into two at a splitting position specific to the firefly luciferase, and 58 to 78 amino acid residues toward the N-terminal beyond the splitting position. When the N-terminal and C-terminal fragments are bound together, firefly luciferase activity is exhibited.

### CROSS-REFERENCE TO RELATED APPLICATION

The present application is a Continuing Application based on International Application PCT/JP2014/074606 filed on Sep. 10, 2014, the entire disclosure of this earlier application being herein incorporated by reference.

### TECHNICAL FIELD

The present disclosure relates a recombinant protein, a gene encoding the recombinant protein, and an analysis method using the recombinant protein and the gene.

### BACKGROUND

Reporter assay techniques and cell imaging techniques have heretofore been used for observing various biological phenomena. In particular, cell imaging techniques involve the use of genes that encode fluorescent proteins or photoproteins to modify biological cells for the analysis of various biological phenomena in the cells using fluorescence or luminescence as an indicator. Cell imaging techniques that involve the use of fluorescent proteins, however, have the drawbacks of low signal-to-noise ratios due to high levels of background fluorescence as a result of self-fluorescence of cells; a limited range of available measurement targets due to a narrow dynamic range; and so forth.

This led to the recent development of cell imaging techniques that use luciferase-luciferin luminescence. For example, PTL 1 discloses a cell-specific gene expression imaging method that uses split luciferase derived from the North American firefly (Photinus pyralis). PTL 2 discloses a calcium indicator that changes luminescence intensity in a calcium ion concentration-dependent manner. The calcium indicator is produced using a luciferase gene derived from the North American firefly (Photinus pyralis), and calmodulin gene and M13 gene whose products interact with each other in a calcium-dependent manner. PLT 2 also discloses an imaging method using this calcium indicator.

Further, PTL 3 discloses a calcium indicator that changes luminescence intensity in a calcium ion concentration-dependent manner. The calcium indicator is produced using a luciferase gene derived from the North American firefly (Photinus pyralis) and calmodulin gene whose product undergoes conformational changes in a calcium dependent manner. PTL 4 discloses split luciferases having various luminescence intensities produced using luciferase derived from the North American firefly (Photinus pyralis).

### CITATION LIST

### Patent Literature

PTL1: JP2007155558A

PTL2: JP201251824A

PTL3: JP201290635A

PTL4: U.S. Pat. No. 7,601,517B

### Non-Patent Literature

NPL1: Miyawaki A. Llopis J, Heim R, McCaffery J M, Adams I A, Ikura M, Tsien R Y. “Fluorescent indicators for Ca2+ based on green fluorescent proteins and calmodulin.” Nature, vol. 388(6645), pp. 882-887, 1997

### SUMMARY

The split recombinant protein disclosed herein includes:

an N-terminal fragment of a firefly luciferase, the N-terminal fragment being one of two fragments of the firefly luciferase split into two such that activity of the firefly luciferase is restored when the two fragments are bound together;

a C-terminal fragment of a firefly luciferase, the C-terminal fragment including 58 to 78 amino acid residues toward the N-terminal beyond a splitting position at which the firefly luciferase can be split into two such that activity of the firefly luciferase is restored when the two fragments are bound together; and

a linker polypeptide,

wherein firefly luciferase activity is exhibited when the N-terminal and C-terminal fragments are bound together.

In the split recombinant protein disclosed herein, the N-terminal and C-terminal fragments may be each derived from a different firefly luciferase of a different firefly species.

In the split recombinant protein disclosed herein, the N-terminal and C-terminal fragments are each preferably derived from a firefly luciferase of a firefly selected from the group consisting of Pyrocoelia matsumurai, Drilaster Kumejimensis, and Stenocladius flavipennis.

In the split recombinant protein disclosed herein, the N-terminal fragment is preferably derived from a firefly luciferase of Pyrocoelia matsumurai.

In the split recombinant protein disclosed herein, it is preferred that the N-terminal fragment be positioned on the C-terminal side in the split recombinant protein and the C-terminal fragment on the N-terminal side in the split recombinant protein.

The split recombinant protein disclosed herein preferably further includes, a calcium-binding region and an interaction region that can reversibly bind to or dissociate from the calcium-binding region between the N-terminal and C-terminal fragments. More preferably, the calcium-binding region is derived from calmodulin, and the interaction region is M13 peptide.

The gene disclosed herein encodes the split recombinant protein.

The vector disclosed herein includes a promoter sequence, and the gene expressibly linked to the promoter sequence.

The cell disclosed herein includes the vector.

The disclosed method of analyzing intracellular calcium ions includes:

preparing a cell containing a vector that contains a promoter sequence and a gene that encodes a split recombinant protein, the gene being expressibly linked to the promoter sequence;

adding a firefly luciferin to the cell from outside the cell;

measuring a luminescence level in the cell over time: and

analyzing changes in a calcium ion concentration in the cell based on changes in the luminescence level measured,

wherein the split recombinant protein includes the calcium-binding region and the interaction region. The analysis method can also use two or more vectors each containing a different gene that encodes the split recombinant protein having a different luminescence color. The analysis method can also analyze changes in a calcium ion concentration within a single cell.

The disclosed method of analyzing intracellular gene expression includes:

preparing a cell containing a vector that contains a promoter sequence, a target gene and a gene that encodes the split recombinant protein, the both genes being expressibly linked to the promoter sequence;

adding a firefly luciferin to the cell from outside the cell;

measuring a luminescence level in the cell over time; and

analyzing changes in a expression level of the target gene in the cell based on changes in the luminescence level measured. The analysis method can also use two or more vectors each containing a different gene that encodes the split recombinant protein having a different luminescence color.

The vector set disclosed herein includes:

a first vector containing a first promoter sequence and a first gene that encodes an N-terminal fragment of firefly luciferase is expressibly linked to the first promoter sequence, the N-terminal fragment being one of two fragments of the firefly luciferase split into two such that activity of the firefly luciferase is restored when the two fragments are bound together; and

a second vector containing a second promoter sequence and a second gene that encodes a C-terminal fragment of the firefly luciferase is expressibly linked to another promoter sequence, the C-terminal fragment including 58 to 78 amino acid residues toward an N-terminal beyond a splitting position at which the firefly luciferase can be split into two such that activity of the firefly luciferase is restored when the two fragments are bound together.

In the vector set disclosed herein, the N-terminal and C-terminal fragments may be each derived from a firefly luciferase of a different firefly specie. Either one of the promoter sequences may be an inducible promoter sequence.

The disclosed method of analyzing intracellular gene expression includes:

preparing a cell containing the vector set in which either one of the first and second promoter sequences is an inducible promoter sequence;

adding a firefly luciferin to the cell from outside the cell;

adding to the cell an inducer that stimulates the inducible promoter sequence, from outside the cell;

measuring a luminescence level in the cell over time; and

analyzing changes in the activity of the inducible promoter gene in the cell based on changes in the luminescence level measured.

The cell disclosed herein includes the vector set disclosed herein.

### BRIEF DESCRIPTION OF THE DRAWINGS

In the accompanying drawings:

FIG. 1 shows a schematic illustration for explaining an N-terminal fragment (NLuc) and a C-terminal fragment (CLuc);

FIG. 2 shows a schematic illustration of an example of a split recombinant protein according to an embodiment of the present disclosure, wherein an N-terminal fragment (NLuc) is positioned on the C-terminal side of a C-terminal fragment (CLuc);

FIG. 3 shows firefly luciferase activities of split recombinant proteins according to an embodiment of the present disclosure;

FIG. 4 shows measurement results of the luminescent wavelengths of permutated luciferases in E. coli and HEK293 cells (Experiment 3);

FIG. 5 shows measurement results of an experiment wherein firefly luciferase activity is restored by expressing N-terminal and C-terminal fragments of a split recombinant protein according to an embodiment of the present disclosure within a cell using separate vectors;

FIG. 6 shows images of results of promoter activity imaging in HEK293 cells using a vector set according to an embodiment of the present disclosure;

FIG. 7 shows analysis results of promoter activity in HEK293 cells using a vector set according to an embodiment of the present disclosure;

FIG. 8 shows images of results of promoter activity imaging in neuronal cells using a vector set according to an embodiment of the present disclosure;

FIG. 9 shows analysis results of promoter activity in neuronal cells using a vector set according to an embodiment of the present disclosure;

FIG. 10 shows a schematic illustration of a split recombinant protein (luminescent calcium indicator) according to an embodiment of the present disclosure;

FIG. 11 shows comparison results of luminescence levels of split recombinant proteins (luminescent calcium indicators) according to an embodiment of the present disclosure;

FIG. 12 shows results of calcium calibration of luminescent calcium indicators (Experiment 8);

FIG. 13 shows analysis results of intracellular calcium ion concentrations in HEK293 cells upon ATP stimulation (Experiment 9);

FIG. 14 shows images and graphs of results of luminescence-based calcium imaging in HEK293 cells upon ATP stimulation (Experiment 10); and

FIG. 15 shows images and graphs of results of high-speed. luminescence imaging of changes in calcium ion concentrations in HEK293 cells (Experiment 11).

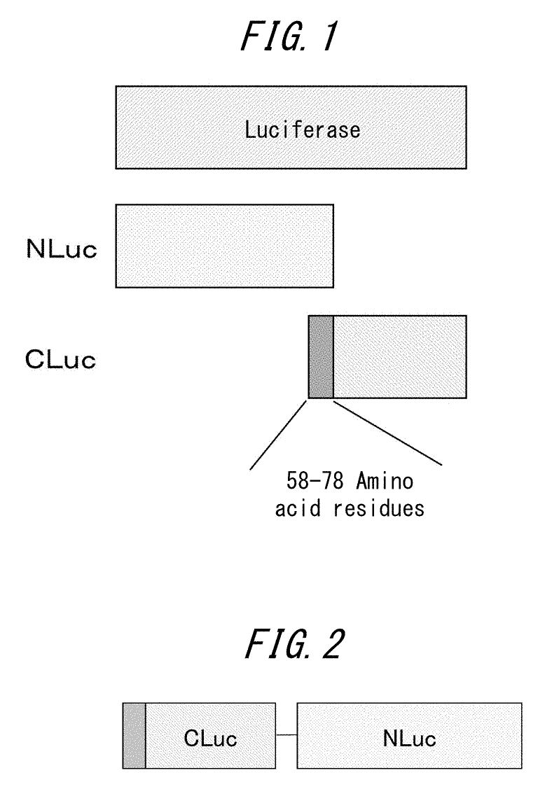

### DETAILED DESCRIPTION

The present disclosure will now be described in detail based on embodiments.

<Split Recombinant Protein>

A split recombinant protein disclosed herein includes:

an N-terminal fragment of firefly luciferase, the N-terminal fragment being one of two fragments of the firefly luciferase split into two such that luciferase activity is restored when the two fragments are bound together;

a C-terminal fragment of the firefly luciferase, the C-terminal fragment including 58 to 78 amino acid residues toward the N-terminal beyond a splitting position at which the firefly luciferase can be split into two such that luciferase activity is restored when the two fragments are bound together; and

a linker polypeptide that links together the N-terminal and C-terminal fragments,

wherein firefly luciferase activity is restored when the N-terminal and C-terminal fragments are bound together.

Luciferase generally refers to a class of enzymes that catalyze a chemical reaction that generates luminescence. The substance that serves as the substrate for the enzyme is called luciferin. Luminescence occurs when luciferin undergoes a chemical change by the catalytic action of luciferase in the presence of adenosine tri phosphate (ATP). Currently available luciferases are those from fireflies and bacteria, but they greatly differ for example in their protein structure and substrate. The present disclosure is directed to firefly luciferase, which is luciferase derived from firefly. The substrate for firefly luciferase is firefly luciferin. Preferably, the firefly luciferin is D-luciferin. By “luciferase activity” is meant an ability to trigger luminescence by catalyzing chemical changes in the substrate luciferin in the presence of ATP. Namely, by “firefly luciferase activity” is meant an ability to trigger luminescence by catalyzing chemical changes in the substrate firefly luciferin in the presence of ATP.

Luciferases are known that are deactivated when split at a specific position into two N-terminal and C-terminal fragments which do not exhibit luciferase activity alone, but can restore luciferase activity once the fragments are bound together to reconstruct the protein (see, e.g., PTL 1 and PTL 4). The N-terminal and C-terminal fragments of luciferase split in this way are also collectively called “split luciferase.” The term “bind” and any modified form thereof as used in the context of the present disclosure refers to binding by any bond, which may be accomplished by a covalent bond, or a noncovalent bond such as ionic bond, hydrogen bond, Van der Waals force or hydrophobic interaction. Binding also may encompass states wherein two or more “bound” targets come close, contact, associate or interact with each other. Specifically, the “binding” between the N-terminal and C-terminal fragments may be accomplished by any bond, and may be accomplished by a covalent bond or a noncovalent bond such as ionic bond, hydrogen bond, Van der Waals force or hydrophobic interaction. The mode of “binding” between the N-terminal and C-terminal fragments may encompass modes wherein the two fragments come close, contact, associate or interact with each other to an extent that they may play a function of luciferase. It should be noted however that modes of binding wherein the N-terminal and C-terminal fragments have only a linkage via a linker peptide are excluded.

By “firefly” is meant an insect that belongs to the Arthropoda: Insecta: Coleoptera: Lampyridae. Any species of firefly can be used for luciferases usable in the present disclosure. Examples of fireflies include Pyrocoelia matsumurai, Drilaster Kumejimensis, Stenocladius flavipennis, North American firefly (Photinus pyralis), Luciola cruciata, Luciola Lateralis, East European firefly (Luciola mingrelica), and Lampyris noctiluca, with Pyrocoelia matsumurai, Drilaster Kumejimensis, and Stenocladius flavipennis being preferred.

The amino acid sequence of firefly luciferase is generally unique to each species of firefly from which the firefly luciferase is derived and thus may differ depending on the species of firefly from which the firefly luciferase is derived. The “splitting position” at which firefly luciferase is split into two N-terminal and C-terminal fragments such that luciferase activity is restored when the fragments are bound together can be at any position so long as it is a position at which the firefly luciferase can be split into two such that luciferase activity is restored when the split fragments are bound together, and may differ depending on the species of firefly from which the firefly luciferase is derived. A person skilled in the art would be able to determine the splitting position in firefly luciferase using any of the methods known in the art. Any information known in the art can also be utilized as to the splitting position. The amino acid sequences of the “N-terminal fragment” and “C-terminal fragment” included in the split recombinant protein disclosed herein may also differ depending on the species of firefly from which firefly luciferase is derived.

The “N-terminal fragment” included in the split recombinant protein disclosed herein is an N-terminal fragment of firefly luciferase split into two such that luciferase activity is restored when the two fragments are bound together, and has an amino acid sequence from residue 1 to the N-terminal side residue adjacent to the “split position” of the firefly luciferase. The “C-terminal fragment” included in the split recombinant protein disclosed herein may derived from firefly luciferase of a firefly which is different from the firefly from which the firefly luciferase for the N-terminal fragment is derived. The “C-terminal fragment” is a C-terminal fragment which contains 58 to 78 amino acid residues toward the N-terminal beyond a splitting position at which the firefly luciferase can be split into two such that firefly luciferase activity is restored when the fragments are bound together. These “N-terminal fragment” and “C-terminal fragment” may be derived from wide-type firefly luciferase and may also have a mutation such as substitution, deletion or addition of one or more amino acid residues to an extent that firefly luciferase activity can be restored when the fragments are bound together.

For example, wide-type firefly luciferase derived from Pyrocoelia matsumurai consists of 562 amino acids encoded by a nucleotide sequence having SEQ ID NO: 1, and has an amino acid sequence having SEQ ID NO: 2. By way of one example, the firefly luciferase can be split into an N-terminal fragment having residues 1 to 416 (SEQ ID NO: 3) and a C-terminal fragment having residues 417 to 562 (SEQ ID NO: 4) such that firefly luciferase activity can be restored when the fragments are bound together. Thus, the “N-terminal fragment” derived from Pyrocoelia matsumurai included in the split recombinant protein disclosed herein may have an amino acid sequence from residues 1 to 416 of wide-type luciferase of Pyrocoelia matsumnurai. The “C-terminal fragment” derived from Pyrocoelia matsumurai included in the split recombinant protein disclosed herein may have an amino acid sequence from any one of residues 339 to 359 to residue 562 of wide-type luciferase of Pyrocoelia matsumurai.

Wide-type firefly luciferase derived from Drilaster Kumejimensis consists of 547 amino acids encoded by a nucleotide sequence having SEQ ID NO: 5, and has an amino acid sequence having SEQ ID NO: 6. By way of one example, the firefly luciferase can be split into an N-terminal fragment having residues 1 to 416 (SEQ ID NO: 7) and a C-terminal fragment having residues 417 to 547 (SEQ ID NO: 8) such that firefly luciferase activity can be restored when the fragments are bound together. Thus, the “N-terminal fragment” derived from Drilaster Kumejimensis included in the split recombinant protein disclosed herein may have an amino acid sequence from residues 1 to 416 of wide-type luciferase of Drilaster Kumejimensis. The “C-terminal fragment” derived from Drilaster Kumejimensis included in the split recombinant protein disclosed herein may have an amino acid sequence from any one of residues 339-359 to residue 547 of wide-type luciferase of Drilaster Kumejimensis.

Wide-type firefly luciferase derived from Stenocladius flavipennis consists of 555 amino acids encoded by a nucleotide sequence having SEQ ID NO: 9, and has an amino acid sequence having SEQ ID NO: 10. By way of one example, the firefly luciferase can be split into an N-terminal fragment having residues 1 to 424 (SEQ ID NO: 11) and a C-terminal fragment having residues 425 to 555 (SEQ ID NO: 12) such that firefly luciferase activity can be restored when the fragments are bound together. Thus, the “N-terminal fragment” derived from Stenocladius flavipennis included in the split recombinant protein disclosed herein may have an amino acid sequence from residue 1 to 424 of wide-type luciferase of Stenocladius flavipennis. The “C-terminal fragment” derived from Stenocladius flavipennis included in the split recombinant protein disclosed herein may have an amino acid sequence from any one of residues 347-367 to residue 555 of wide-type luciferase of Stenocladius flavipennis.

Wide-type firefly luciferase derived from the North American firefly (Photinus pyralis) consists of 550 amino acids. By way of one example, the firefly luciferase can be split into an N-terminal fragment having residues 1 to 416 and a C-terminal fragment having residues 417 to 550 such that firefly luciferase activity can be restored when the fragments are bound together. Thus, the “N-terminal fragment” derived from the North American firefly included in the split recombinant protein disclosed herein may have an amino acid sequence from residues 1 to 416 of wide-type luciferase of the North American firefly. The “C-terminal fragment” derived from the North American firefly included in the split recombinant protein disclosed herein may have an amino acid sequence from any one of residues 339-364 to residue 550 of wide-type luciferase of the North American firefly.

The split recombinant protein disclosed herein can restore firefly luciferase activity that provides at least several-fold higher luminescence intensity than that of wide-type firefly luciferase derived from the North American firefly (Photinus pyralis), when the “N-terminal fragment” and “C-terminal fragment” are “bound” together to reconstruct the protein. The N-terminal and C-terminal fragments may be derived from firefly luciferase of the same or different species of firefly. The N-terminal and C-terminal fragments of the luciferase can be derived from any combination of fireflies, but are each preferably selected from the group consisting of Pyrocoelia matsumurai, Drilaster Kumejimensis, and Stenocladius flavipennis. The N-terminal fragment can be derived from any species of firefly but is preferably derived from Pyrocoelia matsumurai.

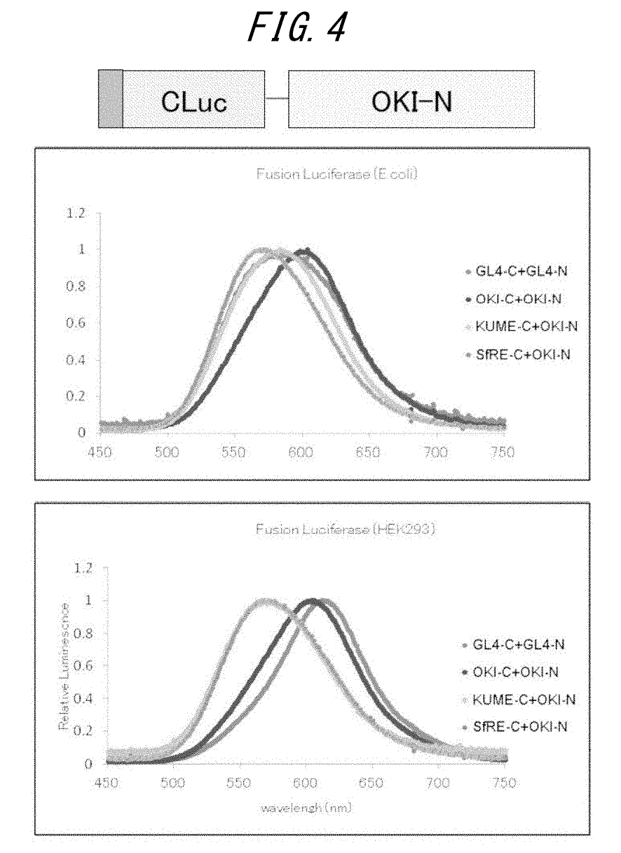

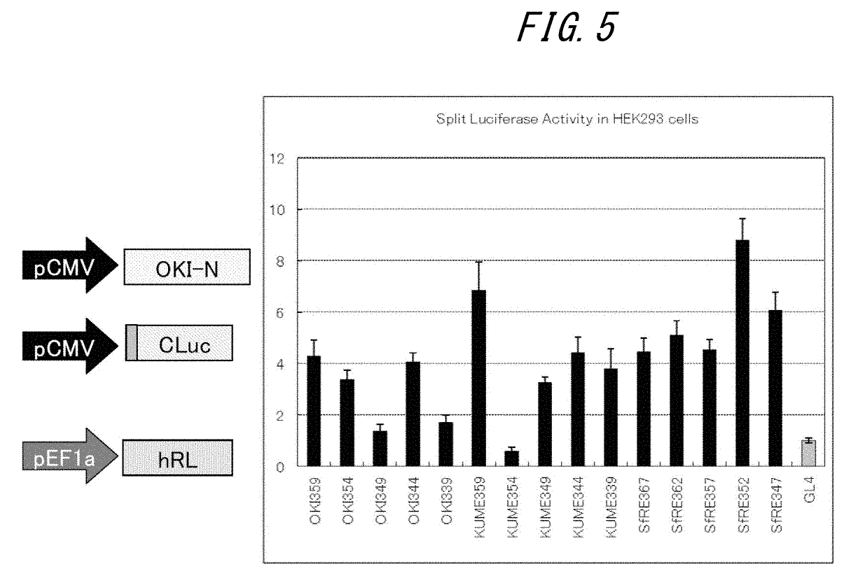

The restored firefly luciferase activity provides luminescence with different luminescence colors, i.e., different peak wavelengths, depending on the species of firefly from which the N-terminal and C-terminal fragments of firefly luciferase are derived, the number of N-terminal side amino acid residues of the C-terminal fragment, or combinations thereof. A specific example thereof is demonstrated in Experiment 3 in Examples (FIG. 4).

The split recombinant protein disclosed herein includes a linker peptide in addition to the N-terminal and C-terminal fragments disclosed herein. Any number and any type of amino acids can be used for the linker peptide so long as the linker peptide can link together the N-terminal and C-terminal fragments disclosed herein, or the N-terminal or C-terminal fragment disclosed herein and another protein, polypeptide or fragment thereof having some function, without compromising their function. By “split” is meant that “N-terminal fragment” and “C-terminal fragment” do not exhibit firefly luciferase activity alone but can restore firefly luciferase activity once they are bound together to reconstruct the protein.

In the split recombinant protein disclosed herein, the N-terminal and C-terminal fragments may assume the original position in firefly luciferase, i.e., the N-terminal fragment may be positioned on the N-terminal side of the C-terminal fragment. Alternatively, the N-terminal and C-terminal fragments may be circularly permutated from the original position in firefly luciferase, i.e., the N-terminal fragment may be positioned on the C-terminal side of the C-terminal fragment.

The split recombinant protein disclosed herein can include one or more proteins, polypeptides or fragments thereof having some function, in addition to the N-terminal fragment, C-terminal fragment and linker peptide disclosed herein. Such proteins are generally also called fusion proteins. In the present disclosure, a peptide portion composed of the one or more proteins, polypeptides or fragments thereof is also referred to as a “region.” Examples of such a functional region include, but not limited to, calcium-binding regions, cyclic AMP-binding regions, cyclic GMP-binding regions, and interaction regions that interact with these binding regions. These regions are preferably positioned between the N-terminal and C-terminal fragments.

By way of one example, the following describes a split recombinant protein that includes a calcium-binding region and an interaction region that can interact with the calcium-binding region. By “calcium-binding region” is meant a peptide portion that can reversibly bind to or dissociate from calcium ion (Ca²⁺) within a cell. By “interaction region” that can interact with a calcium-binding region is meant a peptide portion that can reversibly bind to or dissociate from a calcium-binding region within a cell. Examples of calcium-binding regions include calmodulin (CaM) or fragments thereof. The interaction region can bind to a calcium-binding region bound to calcium ion, and can dissociate from a calcium-binding region from which calcium ion has dissociated. Examples of interaction regions that can interact with a calcium-binding region include M13 peptide. A split recombinant protein that includes a calcium-binding region and an interaction region is expressed for example within a cell, whereby intracellular calcium ion dynamics can be analyzed.

As the calcium-binding region and interaction region, it is possible to use a peptide in which the calcium-binding protein calmodulin and M13 (which binds with calcium-bound calmodulin) are sequentially linked (NPL 1). NPL 1 uses this peptide in which calmodulin and M13 are sequentially linked to produce a calcium sensor protein in which CaM and M13 are flanked by two different fluorescent proteins, and this fluorescent protein is called a “cameleon protein” (hereinafter also referred to as “cameleon”). In particular, as the calcium-binding region and interaction region, it is possible to use a peptide having an amino acid sequence corresponding to residues 230 to 406 of cameleon; a peptide having an amino acid sequence corresponding to residues 230 to 396 of cameleon; a peptide having an amino acid sequence corresponding to residues 230 to 401 of cameleon, a peptide having an amino acid sequence corresponding to residues 230 to 411 of cameleon; or a peptide having an amino acid sequence corresponding to residues 230 to 416 of cameleon. These peptides with specific amino acid sequences can be used for the split recombinant protein disclosed herein even when the peptides have mutations for example for improving sensitivity so long as their function is not compromised.

<Gene>

The present disclosure also relates to a gene that encodes a split recombinant protein, a gene that encodes an N-terminal fragment, and a gene that encodes a C-terminal fragment. In the present disclosure, a “gene” that encodes a protein, polypeptide or fragment thereof may be a DNA or RNA strand, and is intended to mean a DNA or RNA strand having a nucleotide sequence capable of expressing the protein, polypeptide or fragment thereof within a cell or other target. The DNA and RNA strands may consist only of a nucleotide sequence that encodes an amino acid sequence of the protein, polypeptide or fragment thereof, or may include, in addition to the nucleotide sequence, additional nucleotide(s) so long as the function and expression of the protein are not compromised. These nucleotide sequences may have a mutation such as substitution, deletion or addition of one or more nucleotides so long as the function and expression of the protein are not compromised.

<Vector>

The vector disclosed herein is an expression vector containing a gene that encodes a split recombinant protein, a gene that encodes an N-terminal fragment, or a gene that encodes a C-terminal fragment in such a manner that the split recombinant protein, the N-terminal fragment or the C-terminal fragment can be expressed. By “expression vector” is meant a vector in which a gene to be expressed (e.g., a gene that encodes a split recombinant protein, a gene that encodes an N-terminal fragment, a gene that encodes a C-terminal fragment, or a target gene) is expressibly linked. For example, a gene that encodes a split recombinant protein, a gene that encodes an N-terminal fragment, and a gene that encodes a C-terminal fragment are expressibly linked downstream of the promoter region in an expression vector. Any type of vector can be used. Examples of usable vectors include plasmid vectors, phage vectors, and cosmids. A person skilled in the art would be able to select a proper vector based on such conditions as cloning site, promoter sequence, products to be expressed (split recombinant protein, N-terminal fragment, or C-terminal fragment) and/or expression cells in light of common technical knowledge, to construct a desired expression vector in accordance with common procedures. The promoter sequence can also be selected as appropriate in accordance, for example, with the purpose of study. The promoter sequence may be a constitutive promoter sequence, inducible promoter sequence, or tissue-specific promoter sequence.

<Vector Set>

Another embodiment of the present disclosure is directed to a vector set including:

a first vector containing a first promoter sequence and a first gene that encodes an N-terminal fragment of firefly luciferase is expressibly linked to the first promoter sequence, the N-terminal fragment being one of two fragments of the firefly luciferase split into two such that luciferase activity is restored when the two fragments are bound together; and

a second vector containing a second promoter sequence and a second gene that encodes a C-terminal fragment of the firefly luciferase is expressibly linked to the second promoter sequence, the C-terminal fragment including 58 to 78 amino acid residues toward the N-terminal beyond a splitting position at which the firefly luciferase can be split into two such that firefly luciferase activity is restored when the two fragments are bound together.

In this vector set, the N-terminal and C-terminal fragments may be derived from a firefly luciferase of the same or different species of firefly. The vector set may further include another vector in which another N-terminal or C-terminal fragment gene is expressibly linked to another promoter sequence. Further, the vector set can be used in combination with another vector set in which at least one of the gene that encodes an N-terminal fragment, the gene that encodes a C-terminal fragment and the promoter sequence is different. Such a vector set can be constructed as appropriate in accordance with common procedures based on the purpose of study and/or conditions in light of common technical knowledge. One of the promoter sequences used in the vector set may be an inducible promoter sequence. Such a vector set can be used for example in the analysis methods of intracellular gene expression described below.

<Cell>

The cell disclosed herein contains the vector disclosed herein, i.e., a vector in which a promoter sequence, and a gene that encodes a split recombinant protein, a gene that encodes an N-terminal fragment or a gene that encodes a C-terminal fragment are expressibly linked; or the vector set described above. The cell disclosed herein may be any cell so long as a product to be expressed from an expression vector can be expressed therein, and may be animal or plant cell. Usable cells may be of any origin which can be determined as appropriate in accordance, for example, with the purpose of study. Any method can be used to transfer an expression vector into a cell. Examples of transfer methods include transfection, in vitro packaging, freezing and thawing, and electroporation. The transfer method can be determined as appropriate in accordance with the types of vector and cell used.

<Analysis Method>

The phenomenon in which firefly luciferase activity is restored by reconstruction of the split recombinant protein disclosed herein can be used in various analysis methods. For example, the capability of using high luminescence intensity as an indicator and the availability of different luminescence colors enable calcium ion behavior, expression of target genes and promoter sequences, protein-protein interactions, receptor-receptor interactions and other like events in living cells to be analyzed highly precisely either on a single-cell basis or in comparison with a plurality of cells or genes. Thus, the analysis method disclosed herein can be used for imaging, single-cell imaging, high-speed imaging, multicolor imaging and other techniques in cells with weak expression of foreign genes.

An example of an embodiment of the analysis method will now be described.

<Method of Analyzing Intracellular Calcium Ions>

An embodiment of the present disclosure is directed to a method of analyzing intracellular calcium ions which includes:

preparing a cell containing a vector that contains a promoter sequence and a gene that encodes a split recombinant protein, the gene being expressibly linked to the promoter sequence;

adding firefly luciferin to the cell from outside the cell;

measuring the luminescence level in the cell; and

analyzing the calcium ion concentration in the cell based on the luminescence level measured. The split recombinant protein includes a calcium-binding region and an interaction region that interacts with the calcium-binding region. In the present disclosure, the split recombinant protein is also referred to as a luminescent calcium indicator.

In the cell preparation step, the expression vector properly constructed according to the purpose of study and conditions may be transferred into a cell properly selected according to the purpose of study and conditions, as described above. In the cell prepared in this way, a luminescent calcium indicator is expressed. When the promoter sequence is a constitutive promoter sequence, the luminescent calcium indicator is expressed without stimulation such as addition of an inducer. When the promoter sequence is an inducible promoter sequence, the luminescent calcium indicator is expressed in the presence of stimulation such as addition of an inducer. When the promoter sequence is a tissue-specific promoter sequence, the luminescent calcium indicator is expressed only within a cell derived from specific tissue.

In the step of adding firefly luciferin to the prepared cell from outside the cell, addition of firefly luciferin from outside the cell allows the firefly luciferin to penetrate through the plasma membrane into the cell. In the presence of firefly luciferin, the luminescent calcium indicator can restore or lose its firefly luciferase activity according to the presence of calcium ions. It is the conformation of the luminescent calcium indicator, as an entire protein, that determines whether firefly luciferase activity is restored or lost in the presence of calcium ions.

By way of one example, a luminescent calcium indicator will be described that includes, in the order from the N-terminal, a C-terminal fragment, a CaM-M13 fragment of cameleon (residues 230 to 406), and an N-terminal fragment. In the absence of calcium ions; the C-terminal and N-terminal fragments moderately come close with each other and bind together to restore firefly luciferase activity to produce luminescence. On the other hand, in the presence of calcium ions, the calcium ions bind to the calcium-binding region (CaM) and then the interaction region (M13) binds with CaM, resulting in conformational changes of the luminescent calcium indicator as an entire protein to lose firefly luciferase activity and therefore luminescence. Based on such a mechanism, the intracellular calcium ion concentration is reflected by the luminescence level of firefly luciferase.

Based on the measured luminescence level, the intracellular calcium ion concentration can be analyzed. Measurements of luminescence level over time enables changes in intracellular calcium ion concentrations to be analyzed using changes in luminescence levels as an indicator. Any of the commonly used devices and analysis software can be used for the measurement of luminescence level and for the analysis based on the measured luminescence level. Measurements may be made by image capturing. Examples of such devices include luminometers, luminescence microscopes, luminescence imagers, and luminescence detectors.

<Method of Analyzing Intracellular Gene Expression>

Another embodiment of the present disclosure is directed to a method of analyzing intracellular gene expression which includes:

preparing a cell containing an expression vector that contains a target gene and a gene that encodes a split recombinant protein;

adding firefly luciferin to the cell from outside the cell;

measuring the luminescence level in the cell; and

analyzing the expression level of the target gene in the cell based on the luminescence level measured.

In the cell preparation step, the expression vector properly constructed according to the purpose of study and conditions may be transferred into a cell properly selected according to the purpose of study and conditions, as described above. The target gene may be a promoter sequence. In the cell prepared in this way, a target gene and a split recombinant protein are expressed. The target gene may be included in the gene that encodes the split recombinant protein. When the promoter sequence is a constitutive promoter sequence, the target gene and the split recombinant protein are expressed without stimulation such as addition of an inducer. When the promoter sequence is an inducible promoter sequence, the target gene and the split recombinant protein are expressed in the presence of stimulation such as addition of an inducer. When the promoter sequence is a tissue-specific promoter sequence, the target gene and the split recombinant protein are expressed only within a cell derived from specific tissue.

In the step of adding firefly luciferin to the prepared cell from outside the cell, addition of firefly luciferin from outside the cell allows the firefly luciferin to penetrate through the plasma membrane into the cell. When the target gene and the split recombinant protein have been expressed in the presence of firefly luciferin, the split recombinant protein restores firefly luciferase activity and thus luminescence is detected.

Based on the measured luminescence level, the intracellular expression level of the target gene can be analyzed. Measurements of luminescence level over time enables changes in intracellular expression levels to be analyzed using changes in luminescence levels as an indicator. Any of devices and analysis software that can be used for the above-described analysis method of intracellular calcium ions can be used for the measurement of luminescence level and for the analysis based on the measured luminescence level.

In the analysis methods described above, it is possible to use two or more expression vectors containing genes that encode the split recombinant proteins having different luminescence colors. Transfer of such two or more different expression vectors into separate cells and subsequence measurement of the luminescence color unique to each split recombinant protein expressed from the transferred expression vector enables comparison of intracellular calcium ion concentration or intracellular gene expression level between or among the cells. Further, transfer of such two or more different expression vectors into a single cell enables comparison of intracellular calcium ion concentration or intracellular gene expression level within the cell.

<Method of Analyzing Intracellular Gene Expression Using Vector Set>

Another embodiment of the present disclosure is directed to a method of analyzing intracellular gene expression which includes:

preparing a cell containing the vector set in which either one of the promoter sequences is an inducible promoter sequence;

adding firefly luciferin to the cell from outside the cell;

adding to the cell an inducer that stimulates the inducible promoter sequence;

measuring the luminescence level in the cell over time; and

analyzing changes in the activity of the promoter sequence in the cell based on changes in the luminescence level measured.

In the cell preparation step, the expression vector properly constructed according to the purpose of study and conditions may be transferred into a cell properly selected according to the purpose of study and conditions, as described above. In the cell prepared in this way, N-terminal and C-terminal fragments are separately expressed under the regulation of separate promoter sequences. The gene linked to the constitutive promoter sequence is expressed without stimulation such as addition of an inducer. On the other hand, the gene linked to the inducible promoter sequence is expressed to produce an N-terminal or C-terminal fragment in the presence of stimulation such as addition of an inducer. When the promoter sequence is a tissue-specific promoter sequence, the N-terminal or C-terminal fragment is expressed only within a cell derived from specific tissue.

In the step of adding firefly luciferin to the prepared cell from outside the cell, addition of firefly luciferin from outside the cell allows the firefly luciferin to penetrate through the plasma membrane into the cell. When both the N-terminal and C-terminal fragments have been expressed in the presence of firefly luciferin, the split recombinant protein can be reconstructed to restore firefly luciferase activity and thus luminescence is detected. Since one of the promoter sequences is an inducible promoter sequence in the above-described analysis method, either one of the split recombinant protein fragments whose expression is regulated by the inducible promoter sequence is expressed only in the presence of an inducer that stimulates the inducible promoter sequence.

Thus, the above-described analysis method includes adding an inducer that stimulates the inducible promoter sequence. Addition of a proper inducer results in stimulation and thus expression of the inducible promoter sequence; which in turn results in the expression of a split recombinant protein fragment under the regulation of the inducible promoter sequence. As a consequence, both the N-terminal and C-terminal fragments are present within a cell allowing a split recombinant protein to be reconstructed to restore firefly luciferase activity and thus luminescence is detected. Depletion of the inducer for example by intracellular metabolism stops the expression of the inducible promoter sequence and therefore the expression of a split recombinant protein fragment under the regulation of the inducible promoter sequence as well.

In this way it is possible to analyze the intracellular expression level of the inducible promoter sequence based on the luminescence level measured. Measurements of luminescence level over time enables changes in intracellular expression levels of the inducible promoter sequence to be analyzed using changes in luminescence levels as an indicator. Any of devices and analysis software that can be used for the above-described analysis methods of intracellular calcium ions and intracellular gene expression can be used for the measurement of luminescence level and for the analysis based on the measured luminescence level.

In the above-described analysis method, the vector set may further include another vector in which another N-terminal or C-terminal fragment gene is expressibly linked to another promoter sequence. Further, the vector set can be used in combination with another vector set in which at least one of the gene that encodes an N-terminal fragment, the gene that encodes a C-terminal fragment and the promoter sequence is different.

### EXAMPLES

The present disclosure will now be described in detail based on Examples, which however shall not be construed as being limiting in any way. In Examples, a split recombinant protein in which N-terminal and C-terminal fragments are derived from firefly luciferase of the same species of firefly and in which the N-terminal fragment is positioned on the C-terminal side of the C-terminal fragment is also called a permutated firefly luciferase. Further, in Examples, a split recombinant protein in which N-terminal and C-terminal fragments are derived from firefly luciferase of different species of firefly and in which the N-terminal fragment is positioned on the C-terminal side of the C-terminal fragment is also called a heterologous permutated firefly luciferase.

[Pre-Preparation 1: Cloning of N-Terminal Fragment and C-Terminal Fragment Genes of Firefly Luciferase for Preparation of Permutated Firefly Luciferases]

### [Procedure 1]

For the production of N-terminal fragment (NLuc) genes and C-terminal fragment (CLuc) genes of firefly luciferases derived from Pyrocoelia Matsumurai (hereinafter also designated as “OKI”), Drilaster Kumejimensis (hereinafter also designated as “KUME”), and Stenocladius flavipennis (hereinafter also designated as “SfRE”), synthetic oligo DNAs for PCR having the following sequences were prepared.

<Synthetic Oligo DNAs for Preparation of OKI Luciferase Fragments>

OKI-N_Bgl_Fw:
(SEQ ID NO: 13)
5′-AGATCTGAGGACGACCACAAGAACATCGTG-3′
OKI-N_Eco_Rv:
(SEQ ID NO: 14)
5′-GAATTCATCCTTATCGATCAGGGCATTGGT-3′
OKI399_Bam_Fw:
(SEQ ID NO: 15)
5′-GGATCCGCCACCATGAAGGGCTACGTGAACAACCCC-3′
OKI394_Bam_Fw:
(SEQ ID NO: 16)
5′-GGATCCGCCACCATGAAGGGCCCCATGATTATGAAG-3′
OKI389_Bam_Fw:
(SEQ ID NO: 17)
5′-GGATCCGCCACCATGGGAGAGCTGTGCCTGAAGGGC-3′
OKI384_Bam_Fw:
(SEQ ID NO: 18)
5′-GGATCCGCCACCATGGGCGTGAACCAGCGCGGAGAG-3′
OKI379_Bam_Fw:
(SEQ ID NO: 19)
5′-GGATCCGCCACCATGACCAGCAAGACGCTGGGCGTG-3′
OKI374_Bam_Fw:
(SEQ ID NO: 20)
5′-GGATCCGCCACCATGATTGTGGATCTGGATACCAGC-3′
OKI369_Bam_Fw:
(SEQ ID NO: 21)
5′-GGATCCGCCACCATGTTCTTCAGCGCCAAGATTGTG-3′
OKI364_Bam_Fw:
(SEQ ID NO: 22)
5′-GGATCCGCCACCATGGGAAAGGTGGCCCCATTCTTC-3′
OKI359_Bam_Fw:
(SEQ ID NO: 23)
5′-GGATCCGCCACCATGAAGCCAGGCGCCTGCGGAAAG-3′
OKI354_Bam_Fw::
(SEQ ID NO: 24)
5′-GGATCCGCCACCATGCCACGCGGCGACGATAAGCCAGGC-3′
OKI349_Bam_Fw:
(SEQ ID NO: 25)
5′-GGATCCGCCACCATGGCCGTGATTATTACCCCACGCGGC-3′
OKI344_Bam_Fw:
(SEQ ID NO: 26)
5′-GGATCCGCCACCATGACCGAGACCACCAGCGCCGTGATT-3′
OKI339_Bam_Fw:
(SEQ ID NO: 27)
5′-GGATCCGCCACCATGCAGGGCTATGGCCTGACCGAGACC-3′
OKI-C_Xho_Rv:
(SEQ ID NO: 1)
5′-CTCGAGCAGCTTGGACTTCTTGCCCATCGT-3′

<Synthetic Oligo DNAs for Preparation of KUME Luciferase Fragments>

KUME-N_Bgl_Fw:
(SEQ ID NO: 28)
5′-AGATCTGACATGGAAGATAAGAACGTGGTG-3′
KUME-N_Eco_Rv:
(SEQ ID NO: 29)
5′-GAATTCATCCTTGTCGATCAGGGCGTTGGT-3′
KUME399_Bam_Fw:
(SEQ ID NO: 30)
5′-GGATCCGCCACCATGAAGGGCTACGCCAACAACCCC-3′
KUME394_Bam_Fw:
(SEQ ID NO: 31)
5′-GGATCCGCCACCATGAAGGGCGACATGATCATGAAG-3′
KUME389_Bam_Fw:
(SEQ ID NO: 32)
5′-GGATCCGCCACCATGGGCGAGCTGTGCCTGAAGGGC-3′
KUME384_Bam_Fw:
(SEQ ID NO: 33)
5′-GGATCCGCCACCATGGGCCCTCACCAGAAAGGCGAG-3′
KUME379_Bam_Fw:
(SEQ ID NO: 34)
5′-GGATCCGCCACCATGACCAGACAGAGCCTGGGCCCT-3′
KUME374_Bam_Fw:
(SEQ ID NO: 35)
5′-GGATCCGCCACCATGATCATCGACCTGGACACCAGA-3′
KUME369_Bam_Fw:
(SEQ ID NO: 36)
5′-GGATCCGCCACCATGTTCTTCAGCGCCAAGATCATC-3′
KUME364_Bam_Fw:
(SEQ ID NO: 37)
5′-GGATCCGCCACCATGGGCAAGGTGGTGCCATTCTTC-3′
KUME359_Bam_Fw:
(SEQ ID NO: 38)
5′-GGATCCGCCACCATGAAGGCCGGCTCTACAGGCAAG-3′
KUME354_Bam_Fw:
(SEQ ID NO: 39)
5′-GGATCCGCCACCATGCCCGAGGGCGAAGATAAGGCCGGC-3′
KUME349_Bam_Fw:
(SEQ ID NO: 40)
5′-GGATCCGCCACCATGGCCGTGATCATCACACCCGAGGGC-3′
KUME344_Bam_Fw:
(SEQ ID NO: 41)
5′-GGATCCGCCACCATGACAGAGACAACCAGCGCCGTGATC-3′
KUME339_Bam_Fw:
(SEQ ID NO: 42)
5′-GGATCCGCCACCATGCAGGGCTACGGACTGACAGAGACA-3′
KUME-C_Xho_Rv:
(SEQ ID NO: 43)
5′-CTCGAGCATCTTGCTCTGGGGCCGCTTCAG-3′

<Synthetic Oligo DNAs for Preparation of SfRE Luciferase Fragments>

SfRE-N_Bgl_Fw:
(SEQ ID NO: 44)
5′-AGATCTGCCAGCAGCATGATGAGCAAGAAG-3′
SfRE-N_Eco_Rv:
(SEQ ID NO: 45)
5′-GAATTCATCCTTGTCGATCATCTCGICGGT-3′
SfRE407_Bam_Fw:
(SEQ ID NO: 46)
5′-GGATCCGCCACCATGATGGGCTACTGCAACAACAAG-3′
SfRE402_Bam_Fw:
(SEQ ID NO: 47)
5′-GGATCCGCCACCATGAAGGGCGACATGATCATGATG-3′
SfRE397_Bam_Fw:
(SEQ ID NO: 48)
5′-GGATCCGCCACCATGGGAGAACTCTACCTGAAGGGC-3′
SfRE392_Bam_Fw:
(SEQ ID NO: 49)
5′-GGATCCGCCACCATGGGCCCTCACCAGAGGGGAGAA-3′
SfRE387_Bam_Fw:
(SEQ ID NO: 50)
5′-GGATCCGCCACCATGAGCGGCAAGAGCGTGGGCCCT-3′
SfRE382_Bam_Fw:
(SEQ ID NO: 51)
5′-GGATCCGCCACCATGATCGTGGACCTGAACAGCGGC-3′
SfRE377_Bam_Fw:
(SEQ ID NO: 52)
5′-GGATCCGCCACCATGTTCTTCAGCGCCAAGATCGTG-3′
SfRE372_Bam_Fw:
(SEQ ID NO: 53)
5′-GGATCCGCCACCATGGGCAAGGTGGTGCCATTCTTC-3′
SfRE367_Bam_Fw:
(SEQ ID NO: 54)
5′-GGATCCGCCACCATGAAGCCTGGCTCTACAGGCAAG-3′
SfRE362_Bam_Fw:
(SEQ ID NO: 55)
5′-GGATCCGCCACCATGCCCGAGGGCGAGGATAAGCCTGGC-3′
SfRE357_Bam_Fw:
(SEQ ID NO: 56)
5′-GGATCCGCCACCATGGCCGTGATCATCACCCCCGAGGGC-3′
SfRE352_Bam_Fw:
(SEQ ID NO: 57)
5′-GGATCCGCCACCATGACCGAGACAACCAGCGCCGTGATC-3′
SfRE347_Bam_Fw:
(SEQ ID NO: 58)
5′-GGATCCGCCACCATGCAGGGCTACGGCCTGACCGAGACA-3′
SfRE-C_Xho_Rv:
(SEQ ID NO: 59)
5′-CTCGAGCACTTGCTCTGGGGTTTCTTCAG-3′

[Procedure 2] (PCR Cloning of Firefly Luciferase Fragments)

Using the OKI gene as a template and the above-described synthetic oligo DNAs as primers, the following genes for production of permutated firefly luciferases were amplified by PCR: N-terminal fragment gene (OKI-N: including a nucleotide sequence encoding residues 1 to 416 of OKI-derived luciferase), and C-terminal fragment genes encoding C-terminal fragments with different numbers of amino acid residues (OKI399: including a nucleotide sequence encoding residues 399 to 562 of OKI-derived luciferase; OKI394: including a nucleotide sequence encoding residues 394 to 562 of OKI-derived luciferase; OKI389: including a nucleotide sequence encoding residues 389 to 562 of OKI-derived luciferase; OKI384: including a nucleotide sequence encoding residues 384 to 562 of OKI-derived luciferase; OKI379: including a nucleotide sequence encoding residues 379 to 562 of OKI-derived luciferase; OKI374: including a nucleotide sequence encoding residues 374 to 562 of OKI-derived luciferase; OKI369: including a nucleotide sequence encoding residues 369 to 562 of OKI-derived luciferase; OKI364: including a nucleotide sequence encoding residues 364 to 562 of OKI-derived luciferase; OKI359: including a nucleotide sequence encoding residues 359 to 562 of OKI-derived luciferase; OKI354: including a nucleotide sequence encoding residues 354 to 562 of OKI-derived luciferase; OKI349: including a nucleotide sequence encoding residues 349 to 562 of OKI-derived luciferase; OKI344: including a nucleotide sequence encoding residues 344 to 562 of OKI-derived luciferase and OKI339: including a nucleotide sequence encoding residues 339 to 562 of OKI-derived luciferase).

Using the KUME gene as a template and the above-described synthetic oligo DNAs as primers, the following genes for production of permutated firefly luciferases were amplified by PCR: N-terminal fragment gene (KUME-N: including a nucleotide sequence encoding residues 1 to 416 of KUME-derived luciferase), and C-terminal fragment genes that encode C-terminal fragments with different numbers of amino acid residues (KUME399: including a nucleotide sequence encoding residues 399 to 547 of KUME-derived luciferase; KUME394: including a nucleotide sequence encoding residues 394 to 547 of KUME-derived luciferase; KUME389: including a nucleotide sequence encoding residues 389 to 547 of KUME-derived luciferase; KUME384: including a nucleotide sequence encoding residues 384 to 547 of KUME-derived luciferase; KUME379: including a nucleotide sequence encoding residues 379 to 547 of KUME-derived luciferase; KUME374: including a nucleotide sequence encoding residues 374 to 547 of KUME-derived luciferase; KUME369: including a nucleotide sequence encoding residues 369 to 547 of KUME-derived luciferase; KUME364: including a nucleotide sequence encoding residues 364 to 547 of KUME-derived luciferase; KUME359: including a nucleotide sequence encoding residues 359 to 547 of KUME-derived luciferase; KUME354: including a nucleotide sequence encoding residues 354 to 547 of KUME-derived luciferase; KUME349: including a nucleotide sequence encoding residues 349 to 547 of KUME-derived luciferase; KUME344: including a nucleotide sequence encoding residues 344 to 547 of KUME-derived luciferase; and KUME339: including a nucleotide sequence encoding residues 339 to 547 of KUME-derived luciferase).

Using the SfRE gene as a template and the above-described synthetic oligo DNAs as primers, the following genes for production of permutated firefly luciferases were amplified by PCR: N-terminal fragment gene (SfRE-N: including a nucleotide sequence encoding residues 1 to 424 of SfRE-derived luciferase), and C-terminal fragment genes that encode C-terminal fragments with different numbers of amino acid residues (SfRE407: including a nucleotide sequence encoding residues 407 to 555 of SfRE-derived luciferase; SfRE402: including a nucleotide sequence encoding residues 402 to 555 of SfRE-derived luciferase; SfRE397: including a nucleotide sequence encoding residues 397 to 555 of SfRE-derived luciferase; SfRE392: including a nucleotide sequence encoding residues 392 to 555 of SfRE-derived luciferase; SfRE387: including a nucleotide sequence encoding residues 387 to 555 of SfRE-derived luciferase; SfRE382: including a nucleotide sequence encoding residues 382 to 555 of SfRE-derived luciferase; SfRE377: including a nucleotide sequence encoding residues 377 to 555 of SfRE-derived luciferase; SfRE372: including a nucleotide sequence encoding residues 372 to 555 of SfRE-derived luciferase; SfRE367: including a nucleotide sequence encoding residues 367 to 555 of SfRE-derived luciferase; SfRE362: including a nucleotide sequence encoding residues 362 to 555 of SfRE-derived luciferase; SfRE 357: including a nucleotide sequence encoding residues 357 to 555 of SfRE-derived luciferase; SfRE352: including a nucleotide sequence encoding residues 352 to 555 of SfRE-derived luciferase; and SfRE347: including a nucleotide sequence encoding residues 347 to 555 of SfRE-derived luciferase).

[Pre-Preparation 2: Preparation of E. coli Expression Plasmid Encoding Permutated Firefly Luciferase Gene]

### [Procedure]

A PCR-amplified N-terminal fragment gene was ligated between the BglII and EcoRI sites of E. coli expression plasmid pRSET/A (Invitrogen), and a C-terminal fragment gene from the same species of firefly was ligated between the BamHI and XhoI sites to prepare an E. coli expression plasmid encoding a permutated firefly luciferase gene. In this plasmid, the C-terminal fragment gene is positioned on the 5′ side of the N-terminal fragment gene, so that in the expressed permutated firefly luciferase, the N-terminal fragment is positioned on the C-terminal side of the C-terminal fragment and the C-terminal fragment is positioned on the N-terminal side of the N-terminal fragment.

[Experiment 1: Measurement of Luminescence Activity of Permutated Firefly Luciferase Gene]

### [Procedure]

The pRSET vector containing the permutated firefly luciferase gene was transformed into JM109(DE3) strain of E. coli and cultured overnight at 37° C. To 50 μL of culture was added 50 μL of Bright-Glo (Promega) and the culture was allowed to stand for 5 min at room temperature. Luminescence level for 10 sec was then measured using a luminometer (Luminescencer-JNR II, ATTO).

[Results of Experiment 1]

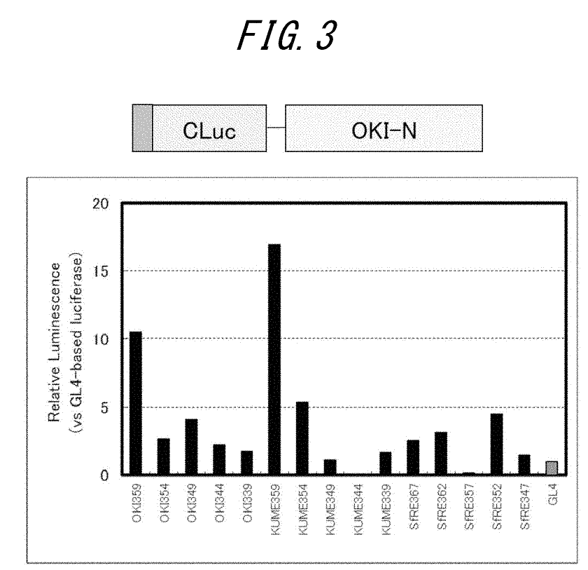

The prepared expression vector was transformed into E. coli (JM109 (DE3)) and cultured overnight in LB medium. To 50 μL of culture was added an equal volume of Bright-Glo and the culture was allowed to stand for 5 min at room temperature. Luminescence level of the reaction solution was reported as counts per 10 sec measured with the luminometer. As a control, permutated luciferase (GL4) derived from the North American firefly (Pholinus pyralis) luciferase was used. In FIG. 3, luminescence activities of GL4-derived and OKI-derived permutated luciferases are shown in terms of relative values with the activity of GL4 being 1. The vertical axis in FIG. 3 represents relative luminescence level. Among different types of permutated luciferase, OKI-derived permutated luciferases OK359, OKI354, OKI349, OKI344 and OKI339 were shown to exhibit high levels of luminescence activity, and OKI349, OKI344 and OKI339 were shown to exhibit higher levels of luminescence activity than GL4. KUME- or SfRE-derived permutated luciferases exhibited no luminescence activity (data not shown).

[Pre-Preparation 3: Preparation of E. coli Expression Plasmid Encoding Heterologous Permutated Luciferase Gene]

### [Procedure]

A PCR-amplified OKI-derived N-terminal fragment (OKI-N) gene was ligated between the BglII and EcoRI sites of E. coli expression plasmid pRSET/A (Invitrogen), and a KUME- or SfRE-derived C-terminal fragment gene was ligated between the BamHI and XhoI sites to prepare an E. coli expression plasmid encoding a heterologous permutated luciferase gene. In this plasmid, the KUME- or SfRE-derived C-terminal fragment gene is positioned on the 5′ side of the OKI-derived N-terminal fragment gene, so that in the expressed permutated firefly luciferase, the OKI-derived N-terminal fragment is positioned on the C-terminal side of the KUME- or SfRE-derived C-terminal fragment and the KUME- or SfRE-derived C-terminal fragment is positioned on the N-terminal side of the OKI-derived N-terminal fragment.

[Experiment 2: Measurement of Luminescence Activity of Heterologous Permutated Luciferase Gene]

### [Procedure]

The pRSET vector containing the heterologous permutated luciferase gene was transformed into JM109(DE3) strain of E. coli and cultured overnight at 37° C. in LB medium. To 50 μL of culture was added 50 μL of Bright-Glo (Promega) and the culture was allowed to stand for 5 min at room temperature. Luminescence level for 10 sec was then measured using a luminometer (Luminescencer-JNR II, ATTO).

[Results of Experiment 2]

An expression vector encoding a heterologous permutated luciferase gene derived from different luciferases was prepared by combining an NLuc gene of OKI and a CLuc gene of KUME or SfRE in a vector. The prepared heterologous permutated luciferase expression vector was transformed into E. coli (JM109 (DE3)) and cultured overnight in LB medium. To 50 μL of culture was added an equal volume of Bright-Glo and the culture was allowed to stand for 5 min at room temperature. Luminescence level of the reaction solution was reported as counts per 10 sec measured with the luminometer. As a control, permutated luciferase (GL4) derived from the North American firefly (Pholinus pyralis) luciferase GL4 was used. In FIG. 3, luminescence activities of heterologous permutated luciferases are shown in terms of relative values with the luminescence activity of GL4 being 1. The vertical axis in FIG. 3 represents relative luminescence level. Heterologous permutated luciferases expressed using a KUME- or SfRE-derived CLuc gene and an OKI-derived NLuc gene were also shown to exhibit strong luminescence activity.

[Pre-Preparation 4: Preparation of Animal Expression Plasmid Encoding Permutated Luciferase Gene]

### [Procedure]

The permutated luciferase gene or heterologous permutated luciferase gene incorporated into pRSET was excised at the BamHI and EcoRI sites, purified, and ligated between the BamHI and EcoRI sites of animal expression plasmid pcDNA3.1 (Invitrogen) to prepare an animal expression plasmid encoding the (heterologous) permutated luciferase gene.

[Experiment 3: Measurement of Luminescence Wavelength of Permutated Luciferase in E. coli and HEK293 Cells]

[Procedure 1] (Measurement of Luminescence Wavelength of Permutated Luciferase Gene in E. coli)

A pRSET vector containing a permutated luciferase gene or heterologous permutated luciferase gene was transformed into JM109(DE3) strain of E. coli and cultured overnight at 37° C. To 100 μL of culture was added 1 mM luciferin and the culture was allowed to stand for 5 min at room temperature. Luminescence wavelength was then measured using LumiFL Spectro Capture (model AB-1850, ATTO).

[Procedure 2] (Culture of HEK293 Cells)

HEK293 cells acquired from American Type Culture Collection (ATCC) were cultured in a 5% CO₂ incubator in Earle's MEM culture medium (GIBCO) supplemented with 10% Fetal Bovine Serum and 1× Nonessential amino acids.

[Procedure 3] (Transfer of Luciferase Fragment Expression Plasmid into HEK293 Cells)

The animal expression plasmid encoding the permutated luciferase gene or heterologous permutated luciferase gene was transferred into the HEK293 cells by electroporation using an electroporator (NEPA21 Super Electroporator, Nepa Gene Co., Ltd.). The transfected HEK293 cells were seeded onto 35 mm-diameter glass bottom dishes at a density of 2×10⁵ cells/dish and cultured overnight in a 5% CO₂ incubator.

[Procedure 4] (Measurement of Active Wavelength of Permutated Luciferase in HEK293 Cells)

1 mM luciferin was added to culture medium and the culture was allowed to stand for 5 min at room temperature. Luminescence wavelength was then measured using LumiFL Spectro Capture (model AB-1850, ATTO).

[Results of Experiment 3]

The measured luminescence wavelengths of permutated luciferases and heterologous permutated luciferases in E. coli and HEK.293 cells are shown in FIG. 4, where the vertical axis represents relative luminescence level and the horizontal axis represents luminescence wavelength. The upper graph shows luminescence wavelengths in E. coli, and the lower graph shows luminescence wavelengths in HEK293 cells. Permutated luciferase based on OKI-C and OKI-N showed maximum luminescence at 603 nm (E. coli, HEK293 cells); heterologous permutated luciferase based on KUME-C and OKI-N at 583 nm (E. coli) and 568 nm (HEK293 cells); heterologous permutated luciferase based on SfRE-C and OKI-N at 568 nm (E. coli, HEK293 cells); and permutated luciferase based on GL4-C and GL4-N at 583 nm (E. coli) and 612 nm (HEK293 cells). These results show that in HEK293 cells the luminescence wavelength of OKI- KUME- and SfRE-based permutated luciferases was blue-shifted by 9-44 nm with respect to the luminescence wavelength of GL4-based permutated luciferase. Further, while the luminescence wavelength of the OKI-based luminescent indicator and SfRE-based luminescent indicator did not change between E. coli and HEK293 cells, the luminescence wavelength of the KUME-based luminescent indicator was blue-shifted in HEK293 cells and the luminescence wavelength of the GL4-based luminescent indicator was red-shifted in HEK.293 cells.

[Pre-Preparation 5: Preparation of Animal Expression Plasmid Encoding Luciferase Fragment Gene]

### [Procedure]

The OKI-derived N-terminal fragment gene incorporated into pRSET was excised at the BglII and EcoRI sites, purified, and ligated between the BamHI and EcoRI sites of animal expression plasmid pcDNA3.1 (Invitrogen) to prepare an animal expression plasmid encoding the luciferase fragment gene. Further, the OKI-derived C-terminal fragment gene, KUME-derived C-terminal fragment gene or SfRE-derived C-terminal fragment gene incorporated in pRSET was excised at the BamHI and XhoI sites, purified, and ligated between the BamHI and XhoI sites of animal expression plasmid pcDNA3.1 (Invitrogen) to prepare an animal expression plasmid encoding the C-terminal fragment.

[Experiment 4: Restoration of Luminescence by Reconstruction of Luciferase Fragments in HEK293 Cells]

[Procedure 1] (Transfer of Luciferase Fragment Expression Plasmids in HEK293 Cells)

The OKI-derived N-terminal fragment expression plasmid and OKI-derived C-terminal fragment expression plasmid; the OKI-derived N-terminal fragment expression plasmid and KUME-derived C-terminal fragment expression plasmid; or the OKI-derived N-terminal fragment expression plasmid and SfRE-derived C-terminal fragment expression plasmid were mixed and transferred into HEK293 cells by electroporation using NEPA21 (Nepa Gene Co., Ltd.), As an internal control for gene transfer, renilla luciferase (hRL) gene whose expression is induced by the human EF1α promoter was used. The transfected HEK293 cells were seeded into a 96-well multiplate at a concentration of 1×10⁴ cells/well and cultured overnight in a 5% CO₂ incubator.

[Procedure 2] (Measurement of Activity of HEK293 Cells Expressing Luciferase Fragments) 1 mM luciferin (Wako Pure Chemical industries, Ltd.) was added to culture medium and the culture was allowed to stand for 15 min at room temperature, and luminescence level for 10 sec was measured using a liminometer (Luminescencer-JNR II, ATTO). Next, 10 μM coelenterazine was added, and luminescence level for 10 sec was measured through a 470-490 nm band-pass filter to correct experimental errors due to variation in gene transfer efficiency among the wells.

[Results of Experiment 4]

To compare luminescence intensities in living cells when luciferase fragments are separately expressed, the OKI-N gene expression plasmid and OKI-C gene expression plasmid; OKI-N gene expression plasmid and KUME-C gene expression plasmid; or OKI-N gene expression plasmid and SfRE-C gene expression plasmid were transferred into REK293 cells, luciferin (final conc.=1 mM) was added, and luminescence intensities were measured. The results are shown in FIG. 5, where the vertical axis represents relative luminescence level with the luminescence level of GL4 being 1. As seen from FIG. 5, in living cells, all the combinations of luciferase fragments except for the combination of OKI-N and KUME354 provided higher luminescent intensities than GL4-based luciferase fragments.

[Pre-Preparation 6: Preparation of Luciferase Fragment Expression Vector in which Expression is Induced by c-fos Promoter]

### [Procedure 1]

For cloning of the c-fos promoter region, synthetic oligo DNAs for PCR were prepared. The nucleotides sequences of the synthetic oligo DNAs are shown below.

(Nucleotide Sequences of Synthetic oligo DNAs for Preparation of c-fos Promoter Region)

c-fos_pro_Fw:
(SEQ ID NO: 60)
5′-AGCTCGAGAGCAGTTCCCGTCAATCCCT-3′
c-fos_pro_Rv:
(SEQ ID NO: 61)
5′-CAAAGCTTTGCAGAAGTCCTAGAACAA-3′

[Procedure 2]

Using genomic DNA of HeLa cells as a template and c-fos_pro_Fw and c-fos_pro-Rv as primers, the human c-fos promoter region was amplified by PCR and subcloned into pBluescript II vector.

[Procedure 3]

Expression vector (pfos/OKI-N) in which the OKI-N gene is expressibly linked to the c-fos promoter region such that expression of OKI-N is induced by the c-fos promoter was constructed in the manner described below. Specifically, the Luc2 gene in pGL4.10 was excised at the HindIII and XbaI sites and the OKI-N gene previously digested at the HindIII, and XbaI sites was ligated into the vector. Next, the c-fos promoter region subcloned into the pBluescript II vector was digested at the XhoI. and HindIII sites and ligated between the XhoI and HindIII sites positioned upstream of the OKI-N gene. In this way, a c-fos promoter-inducible OKI-N gene expression vector (pfos/OKI-N) was prepared.

[Experiment 5: Imaging of Changes in c-fos Promoter Activity by Forskolin Stimulation in HEK293 Cells]

### [Procedure 1]

The first vector in which gene expression is induced by the CMV promoter (pCMV/OKI359) and the second vector in which gene expression is induced by the c-fos promoter (pfos/OKI-N) were transferred into HEK293 cells by electroporation using NEPA21 (Nepa Gene Co., Ltd.). The transfected HEK293 cells were seeded onto 35 mm-diameter glass bottom dishes at a density of 2×10⁵ cells/dish and cultured overnight in a 5% CO₂ incubator.

[Procedure 2]

After overnight culture, the culture medium was exchanged with serum-free CO₂-independent culture medium (Invitrogen) and the culture was incubated for 4 h. Luciferin was then added to a final concentration of and the culture was incubated for an additional 1 h.

[Procedure 3]

The cell-containing culture dish was loaded onto a luminescence microscope (LV-200, Olympus). An EM-CCD camera (iXon, Andor) was used as an image capturing device to acquire luminescence images. The acquired images were transferred to a personal computer. Stimulation was effected using forskolin (final conc.=5 μM), a cAMP synthesis activator. Immediately after stimulation, luminescence images were continuously acquired every 10 min by LV-200. The luminescence images were analyzed using MetaMorph software (Universal Imaging).

[Results of Experiment 5]

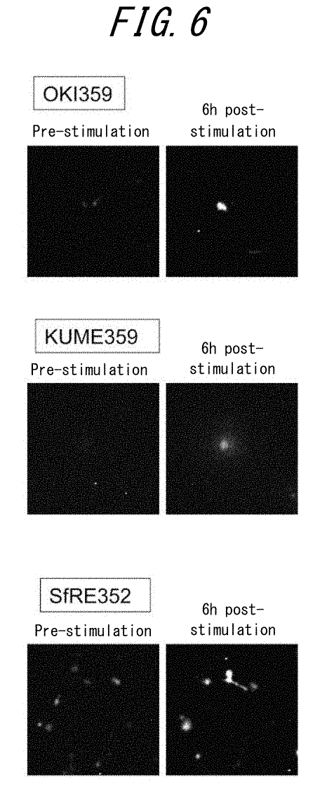

Luminescence images of HEK293 cells transfected with the first expression vector containing a C-terminal luciferase fragment gene whose expression is induced by the CMV promoter (pCMV/OKI359, pCMV/KUME359 or pCMV/SfRE352) and the second expression vector (pfos/OKI-N) are shown in FIG. 6. The left images are luminescence images prior to forskolin stimulation, and the right images are luminescence images taken 6 h after forskolin stimulation.

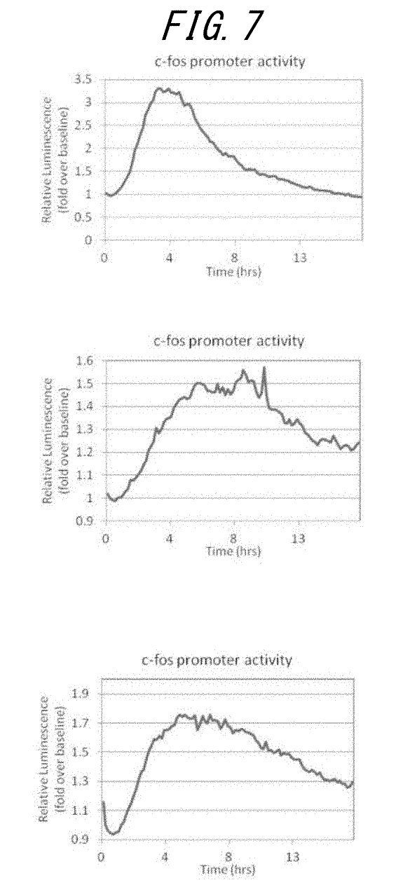

Changes in luminescence intensity (indicative of c-fos promoter activity per cell) associated with forskolin stimulation in HEK293 cells were also analyzed based on the acquired luminescence images. These changes in luminescence intensity are shown in FIG. 7, where the horizontal axis represents time, the vertical axis represents relative luminescence intensity, and the trace represents changes in luminescence intensity in a cell.

As shown in FIG. 7, HEK293 cells were observed to exhibit elevated levels of c-fos promoter activity immediately after forskolin stimulation for every combination of luciferase fragments.

[Pre-Preparation 7: Preparation of Luciferase Fragment Expression Vector in Which Expression is Induced by Synapsin I (SYN) Promoter]

### [Procedure 1]

For cloning of the SYN promoter region, synthetic oligo DNAs for PCR were prepared. The nucleotides sequences of the synthetic oligo DNAs are shown below.

### (Nucleotide Sequences of Synthetic Oligo DNAs for Preparation of SYN Promoter Region)

SYN_pro_Fw:
(SEQ ID NO: 62)
5′-CTCGAGGCCACATTGGCACTGGATGTTTCC-3′
SYN_pro_Rv:
(SEQ ID NO: 63)
5′-AAGCTTGACTTGGGGCAGGGGGTCCTAGGG-3′

[Procedure 2]

Using genomic DNA of HeLa cells as a template and SYN_pro_Fw and SYN_pro-Rv as primers, the human SYN promoter region was amplified by PCR and subcloned into pBluescript II vector.

[Procedure 3]

Expression vector (pSYN/OKI359) in which the OKI359 gene is expressibly linked to the SYN promoter region such that expression of a C-terminal fragment of OKI (OKI359) is induced by the SYN promoter was constructed in the manner described below. Specifically, the Luc2 gene in pGL4.10 was excised at the HindIII and XbaI sites, and the OKI359 gene previously digested at the HindIII and XbaI sites was ligated into the vector. Next, the SYN promoter region subcloned into the pBluescript II vector was digested at the XhoI and HindIII sites and ligated between the XhoI and HindIII sites positioned upstream of the OKI359 gene. In this way, a SYN promoter-inducible OKI359 gene expression vector (pSYN/OKI359) was prepared.

[Experiment 6: Imaging of Changes in c-fos Promoter Activity by Forskolin Stimulation in Rat Brain Slice Culture]

As a specimen in which different types of neuronal cells are mixed, a sliced specimen of the rat hippocampus was used. Specifically, in this measurement, the c-fos promoter activity of neuronal cells was measured under near in vitro conditions.

[Procedure 1]

First, the whole brain was collected from a 7-9 day-old SD rat. A portion of the brain containing the hippocampus was cut into a 400 μm thick slice using LinearSlicer PRO7 (DOSAKA EM). The hippocampus slice was placed on Millicell culture insert (Millipore) and incubated in a 5% CO₂ incubator in Earle's MEM culture medium supplemented with 25% horse serum and 25% Hank's solution. At day 5-7 after initiation of culture, neuronal cells in the rat hippocampus section were transfected with the first expression vector (pSYN/OKI359) and the second expression vector (pfos/OKI-N) by electroporation. The transfected section sample was cultured overnight in a 5% CO₂ incubator in Earle's MEM culture medium supplemented with 25% horse serum and 25% Hank's solution.

[Procedure 2]

After overnight culture, the culture medium was exchanged with serum-free CO₂-independent culture medium (Invitrogen) and the culture was incubated for 4 h. Luciferin was then added to a final concentration of 1 mM and the culture was incubated for an additional 1 h.

[Procedure 3]

A section-containing culture dish was loaded onto a luminescence microscope (LV-200, Olympus). An EM-CCD camera (iXon, Andor) was used as an image capturing device to acquire luminescence images. The acquired images were transferred to a personal computer. Stimulation was effected using forskolin (final conc.=5 μM), a cAMP synthesis activator. Immediately after stimulation, luminescence images were continuously acquired every 10 min by LV-200. The luminescence images were analyzed using MetaMorph software (Universal Imaging).

[Results of Experiment 6]

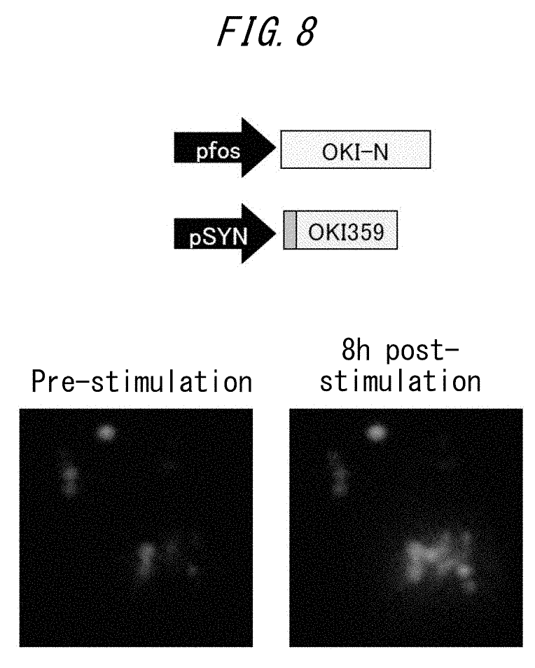

Luminescence images of the section of cultured hippocampus transfected with the first expression vector containing a C-terminal luciferase fragment gene whose expression is induced by the neuronal cell-specific promoter (SYN promoter)(pSYN/OKI359) and the second expression vector (pfos/OKI-N) are shown in FIG. 8. The left image is a luminescence image prior to forskolin stimulation, and the right image is a luminescence image taken 8 h after forskolin stimulation.

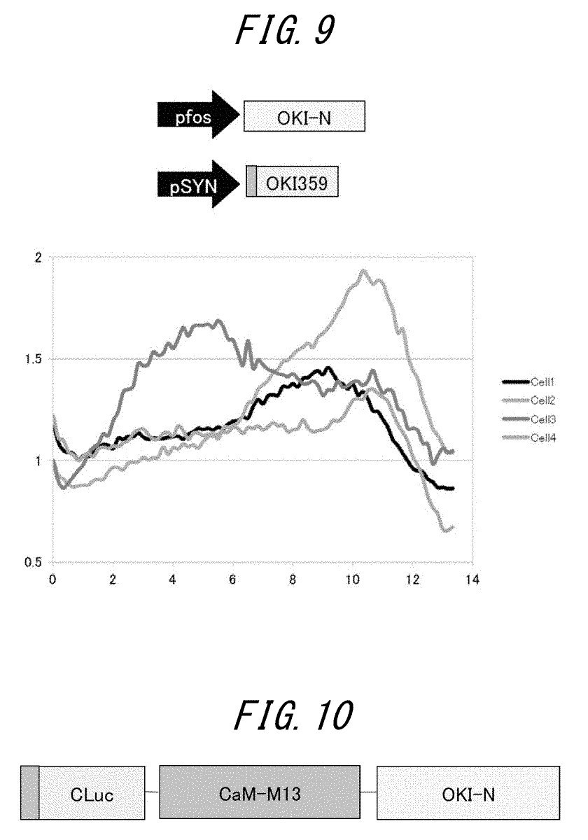

Changes in luminescence intensity (indicative of c-fos promoter activity per cell) associated with forskolin stimulation in neuronal cells were also analyzed based on the acquired luminescence images. The changes in luminescence intensity are shown in FIG. 9, where the horizontal axis represents time, the vertical axis represents relative luminescence intensity, and each trace represents changes in luminescence intensity in each cell.

As shown in FIG. 9, some neuronal cells exhibited elevated levels of c-fos promoter activity immediately after forskolin stimulation, while the others about 6 h after forskolin stimulation. Thus, in the case of neuronal cells, changes in c-fos promoter activity with time after forskolin stimulation differ greatly among cells.

[Pre-Preparation 8: Preparation of Luminescent Calcium Indicator Expression Vector]

### [Procedure 1]

For cloning of calmodulin and M13, synthetic oligo DNAs for PCR having the following nucleotide sequences were prepared.

### [Nucleotide Sequences of Synthetic Oligo DNAs for Preparation of CaM-M13 Gene]

CaM-M13_Fw:
(SEQ ID NO: 64)
5′-GCCCTCGAGCATGACCAACTGACAGAAGAG-3′
CaM-M13_Rv:
(SEQ ID NO: 65)
5′-CATGGATCCCAGTGCCCCGGAGCTGGAGAT-3′

[Procedure 2] (PCR Cloning of CaM-M13 Gene)

Using cDNA of the cameleon gene (YC2.1) (NPL 1) as a template and the above synthetic oligo DNAs as primers, the CaM-M13 gene (including a region encoding an amino acid sequence corresponding to residues 230 to 406 of the cameleon protein) was amplified by PCR to amply a region containing a sequence encoding calmodulin (CaM; calcium-binding protein) and a sequence encoding a peptide (M13) capable of reversibly binding to or dissociating from calmodulin.

[Procedure 3] (Preparation of Luminescent Calcium Indicator Gene Expression Vector)

The PCR-amplified CaM-M13 gene was digested at the XhoI and BamHI sites and ligated between the XhoI and BglII sites of pRSET containing a permutated luciferase gene or a heterologous permutated luciferase gene to prepare an E. coli expression vector encoding a luminescent calcium indicator gene. Further, the luminescent calcium indicator gene was digested at the BamHI and EcoRI sites and ligated between the BamHI and EcoRI sites of animal expression vector pcDNA3.1 (Invitrogen) to prepare an animal expression vector encoding a luminescent calcium indicator gene.

[Experiment 7: Measurement of Luminescence Activity of Luminescent Calcium Indicator]

[Procedure 1] (Transfer of Luminescent Calcium Indicator Expression Vector into HEK293 Cells)

The luminescent calcium indicator expression vector was transferred into HEK293 cells by electroporation using NEPA21 (Nepa Gene Co., Ltd), As an internal control for gene transfer, renilla luciferase (hRL) whose expression is induced by the human EF1α promoter was used. The transfected HEK293 cells were seeded into a 96-well multiplate at a concentration of 1×10⁴ cells/well and cultured overnight in a 5% CO₂ incubator.

[Procedure 2] (Measurement of Luminescence Activity of HEK293 Cells Expressing Luminenscent Calcium Indicator)

1 mM luciferin (Wako Pure Chemical Industries, Ltd.) was added to culture medium and the culture was allowed to stand for 15 min at room temperature, and luminescence level for 10 sec was measured using a luminometer (Luminescencer-JNR II ATTO). Next, 10 μM coelenterazine was added, and luminescence level for 10 sec was measured through a 470-490 nm band-pass filter to correct experimental errors due to variation in gene transfer efficiency among the wells.

[Results of Experiment 7]

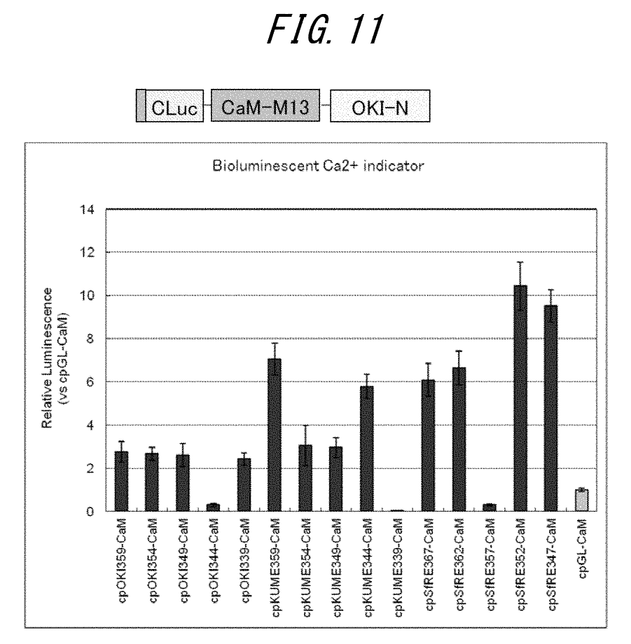

To compare luminescence intensities in mammalian living cells, different luminescent calcium indicator genes were transferred into HEK293 cells and their luminescence level after addition of luciferin (final conc. 1 mM) was measured. The results are shown in FIG. 11, where the vertical axis represents relative luminescence intensity. As seen from FIG. 11, it was discovered that all the luminescent calcium indicators except for cpOKI344-CaM, cpKUME339-CaM and cpSfRE357-CaM exhibited higher luminescence intensities than the GL4-based calcium indicator (cpGL4-CaM) in living cells.

[Experiment 8: Calcium Calibration of Luminescent Calcium Indicator]

### [Procedure 1]

A pRSET vector containing a luminescent calcium indicator gene was transformed into JM109(DE3) strain of E. coli and cultured overnight at 37° C. A His-tagged expressed protein was then purified using QIAexpressionist (QIAGEN).

[Procedure 2]

Protein quantification was performed using Protein Assay Reagent (Bio-Rad), and the protein solution was diluted to a concentration of 1 μg/μL.

[Procedure 3]

To 5 μL of the diluted luciferase solution (5 μg protein) was added 35 μL of 0 μM, 0.017 μM, 0.038 μM, 0.065 μM, 0.10 μM. 0.15 μM, 0.23 μM, 0.35 μM, 0.60 μM, 1.35 μM or 3.50 μM Ca²⁺ buffer solution (Ca²⁺ calibration kit, Invitrogen) and the solution was allowed to stand for 15 min at room temperature.

[Procedure 4]

40 μL of Bright-Glo (Promega) was then added and the solution was allowed to stand for 15 min at room temperature. Luminescence level for 10 sec was then measured at room temperature using a luminometer (Luminescencer-JNR II, ATTO).

[Results of Experiment 8]

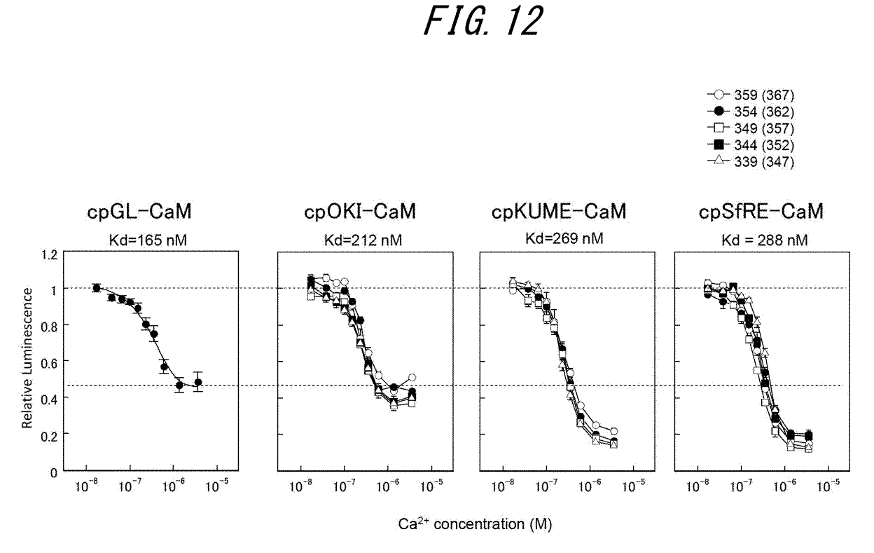

To investigate different luminescent calcium indicators for their sensitivity to calcium, effects of Ca²⁺ concentration in solution on their luminescence activity were investigated using the Ca²⁺ calibration buffer kit. The results are shown in FIG. 12, where the vertical axis represents relative luminescence intensity, and the horizontal axis represents calcium ion concentration. As shown in FIG. 12, the luminescence intensity of every luminescent calcium indictor was shown to decrease with increasing Ca²⁺ concentration.

Further, the luminescent calcium indicators had Kd values for Ca²⁺ in the range of 212-288 nM, indicating that their affinity for Ca²⁺ was slightly lower than that of cpGL4-CaM (Kd value=165 nM). However, these Kd values are comparable with those of high-affinity classes of commercially available Ca²⁺ indicators, indicating that the prepared luminescent calcium indicators are capable of monitoring changes in cytoplasmic Ca²⁺ concentrations. It was also shown that for luminescent calcium indicators containing KUME-C and OKI-N or SfRE-C and OKI-N, a difference in luminescence intensity between the calcium-bound form and calcium-unbound form is large compared to luminescent calcium indicators composed of GL4 or OKI fragments (luminescence intensity ratio of Ca²⁺-bound form to Ca²⁺-unbound form is 1:0.17 for luminescent calcium indicator containing KUME-C and OKI-N; 1:0.16 for luminescent calcium indicator containing SfRE-C and OKI-N; 1:0.42 for luminescent calcium indicator containing OKI; and 1:0.48 for luminescent calcium indicator containing. It was thus shown that luminescent calcium indicators containing KUME-C and OKI-N or SfRE-C and OKI-N may greatly change luminescence intensity with changes in Ca²⁺ concentration.

[Experiment 9: Luminescence Measurement for Changes in Intracellular Calcium Level in HEK293 Cells Upon ATP Stimulation]

### [Procedure 1]

HEK293 cells (1×10⁶ cells) were collected by centrifugation and luminescent calcium indicator expression vectors were transferred into the cells by electroporation using NEPA21. The transfected HEK293 cells were seeded into a 24-well multiplate at a concentration of 1×10⁵ cells/well and cultured overnight in a 5% CO₂ incubator.

[Procedure 2]

After rinsing the cells with CO₂-independent culture medium twice, 200 μL of CO₂-independent culture medium containing D-luciferin (final conc.=2 mM) was added and the plate was allowed to stand for 15 min.

[Procedure 3]

The plate was placed in a luminometer (Luminescencer-JNR II, ATTO), and luminescence intensity for pre- and post-ATP (200 μM) stimulation was measured over time.

[Results of Experiment 9]

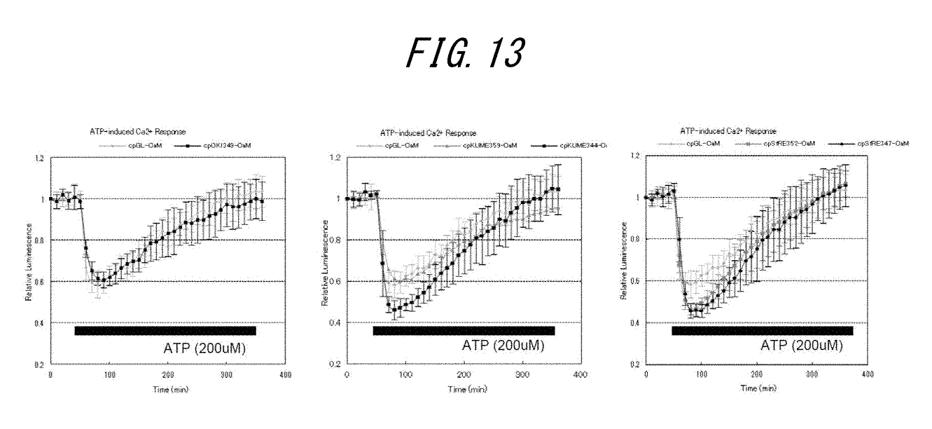

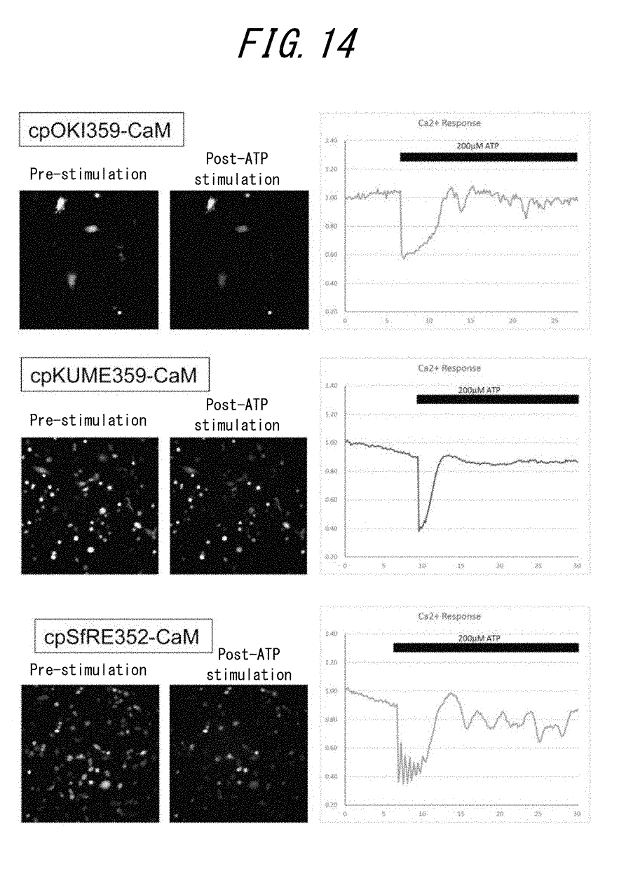

Luminescent calcium indicators were transferred into HEK293 cells and the cells were cultured overnight. The cells were rinsed with CO₂-independent culture medium, luciferin was added to the CO₂-independent culture medium to a final luciferin concentration of 2 mM, and the culture was allowed to stand for 15 min, followed by measurement of luminescence intensity using a luminometer. Luminescence intensity for pre- and post-ATP (200 μM) stimulation was measured over time and plotted in a graph as shown in FIG. 13, where the vertical axis represents relative luminescence intensity and the horizontal axis represents time.

ATP stimulation caused reductions in luminescence intensity for every luminescent calcium indicator, revealing that changes in intracellular calcium ion concentration can be monitored as changes in luminescence intensity. Further, indicators containing a KUME- or SfRE-derived luciferase sequence were shown to exhibit large changes in luminescence intensity in response to changes in calcium ion concentration compared to the GL4-derived indicator. These results are well consistent with the calcium calibration data shown in FIG. 12.

[Experiment 10: Luminescence-Based Calcium Imaging in HEK293 Cells Upon ATP Stimulation]

### [Procedure 1] (Culture of HEK293 Cells)

HEK293 cells were acquired from ATCC and cultured in a 5% CO₂ incubator in Earle's MEM culture medium (GIBCO) supplemented with 10% Fetal Bovine Serum and 1× Nonessential amino acids.

[Procedure 2] (Transfer of Luminescent Calcium Indicator Expression Vector)

The cultured HEK293 cells were seeded onto 35 mm-diameter glass bottom dishes at a density of 2×10⁵ cells/dish and cultured overnight in a 5% CO₂ incubator. Luminescent calcium indicator expression plasmids were transferred into the HEK293 cells by electroporation using NEPA21 and cultured overnight in a 5% CO₂ incubator.

[Procedure 3] (Capturing of Luminescence Images)

2 mM luciferin (Wako Pure Chemical industries, Ltd.) was added to the culture medium and the culture was allowed to stand for 1 h. The culture dish was then loaded onto a luminescence microscope (LV-200, Olympus) and time-lapse image capturing was performed to acquire luminescence images at an interval of 10 sec. Luminescence observation conditions were as follows: magnification of objective lens=40×, exposure time=5 sec, and binning=1×1. An EM-CCD camera (iXon, Andor) was used as a CCD camera, and luminescence images were transferred to a personal computer configured as an image analyzer.

[Results of Experiment 10]

Changes in luminescence intensity of luminescent calcium indicators in response to changes in intracellular calcium ion concentration upon addition of ATP in culture medium were imaged. Luminescence was observed on a single-cell basis when EM gain was set to 1,000 and exposure time to 5 sec. The luminescence images were transferred to the PC every 10 sec. In FIG. 14, the left images are pre-stimulation images, and the right images are post-stimulation images. As shown in FIG. 14, the cells were observed to exhibit decreased levels of luminescence intensity by ATP stimulation. Further, when the luminescence intensity change was plotted versus time, it was observed that the luminescence intensity change caused by ATP stimulation is transient as shown in FIG. 14, where the vertical axis represents relative luminescence intensity with pre-stimulation luminescence intensity being 1, and the horizontal represents time (min). These results show that the luminescent calcium indicators were capable of monitoring intracellular calcium ion concentrations.

[Pre-Preparation 9: Cloning of 5HT2A Receptor Gene]

### [Procedure 1]

For the preparation of the human 5HT2A receptor gene, synthetic oligo DNAs for PCR having the following nucleotide sequences were prepared.

### [Nucleotide Sequences of Synthetic Oligo DNAs for Preparation of Human 5HT2A Receptor Gene]

HU_5HT2AR_Fw:
(SEQ ID NO: 66)
5′-GCCACCATGGATATTCTTTGTGAAGAAAAT-3′
HU_5HT2AR_Rv:
(SEQ ID NO: 67)
5′-TCACACACAGCTCACCTTTTCATTCACTCC-3′

[Procedure 2] (PCR Cloning of Human 5HT2A Receptor Gene)

Using a human brain cDNA library (TAKARA BIO Inc.) as a template and the above synthetic oligo DNAs as primers, a 5HT2A receptor gene (including a region corresponding to the full length of the human 5HT2A receptor gene) was amplified by PCR.

[Procedure 3] (Preparation of Animal Expression Plasmid Encoding Human 5HT2A Receptor Gene]

The cloned gene was incorporated into mammalian expression vector pcDNA3.1(+) (Invitrogen) to prepare a human 5HT2A receptor expression plasmid pcDNA/5HT2AR.

[Experiment 11: High-Speed Luminescence Imaging of Changes in Calcium Ion Concentrations in HEK293 Cells]

### [Procedure 1] (Culture of HEK293 Cells)

HEK293 cells were acquired from ATCC and cultured in a 5% CO₂ incubator in Earle's MEM culture medium (GIBCO) supplemented with 10% Fetal Bovine Serum and 1× Nonessential amino acids.

[Procedure 2] (Transfer of Luminescent Calcium Indicator Expression Vector)

The cells cultured in Procedure 1 were seeded onto 35 mm-diameter glass bottom dishes at a density of 2×10⁵ cells/dish and cultured overnight in a 5% CO₂ incubator. The human 5HT2A receptor expression plasmid pcDNA/5HT2AR and luminescent calcium indicator expression plasmid were transferred into the cells by electroporation using NEPA21 and the cells were cultured overnight in a 5% CO₂ incubator.

[Procedure 3] (Capturing of Luminescence Images)

2 mM luciferin (Promega) was added to the culture medium of the culture obtained in Procedure 2 and the culture was allowed to stand for 1 h. The culture dish was loaded onto a luminescence microscope (LV-200, Olympus) and time-lapse image capturing was performed to acquire luminescence images at an interval of 500 milliseconds. Luminescence observation conditions were as follows: magnification of objective lens:=40×, exposure time=200 milliseconds, and binning=2×2. Using an EM-CCD camera (iXon, Andor), luminescence images were taken and transferred to a personal computer.

[Procedure 4] (Time-Lapse Luminescence Image Capturing After Serotonin Stimulation)

Stimulation by 5HT (final conc.=10 μM) was performed 30 sec after the initiation of the time-lapse image capturing, and time-lapse image capturing was continued.

[Procedure 5]

Regions of interest (ROIs) were designated for each luminescence image captured in Procedure 3 and for each luminescence image captured in Procedure 4. The luminescence intensity of each of the designated ROIs was measured for each luminescence image, and changes in luminescence intensity with time were graphically represented. The luminescence images were analyzed using MetaMorph software (Universal imaging).

[Results of Experiment 11]

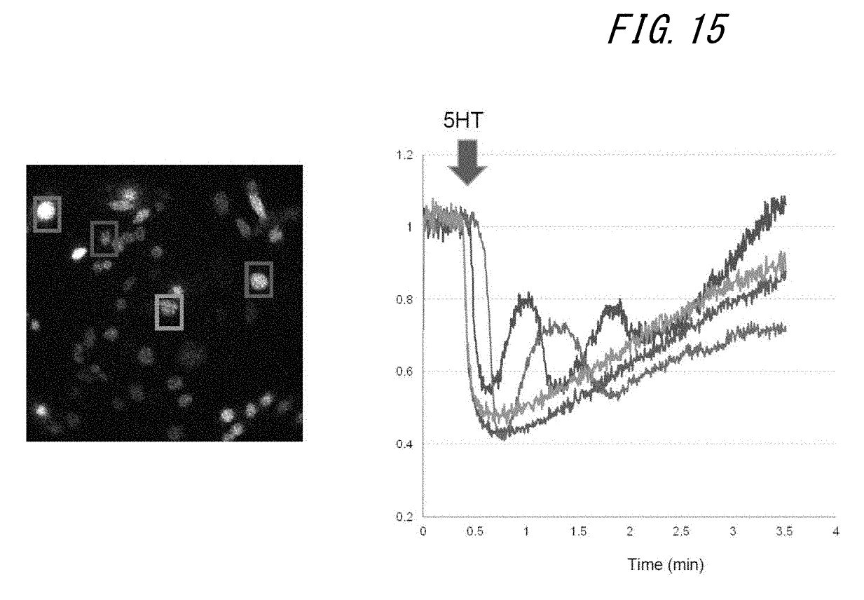

Using a luminescent calcium indicator that has a higher luminescence intensity than a conventional one (cpGL4-CaM), studies were made to confirm whether the luminescent calcium indicator can monitor rapid changes in intracellular concentrations upon receptor stimulation. cpSfRE55-CaM was transferred into 5HT2A receptor-expressing HEK293 cells, and luminescence images were acquired using LV-200. Luminescence was observed on a single-cell basis when EM gain was set to 1,000, exposure time to 200 milliseconds, and binning to 2×2. The cells were stimulated by 5HT (10 μM) while transferring luminescence images to the PC every 500 milliseconds. The results are shown in FIG. 15. In the right graph, the vertical axis represents relative luminescence intensity with pre-stimulation luminescence intensity being 1, and the horizontal represents time (min). Analysis of the pre- and post-stimulation luminescence images revealed the presence of cells that exhibited oscillation, as well as cells that exhibited a transient reduction in luminescence intensity. This result shows that the novel luminescent calcium indicator is also applicable to high-speed calcium imaging where exposure time is 1 sec or less.

As seen from the Examples described above, the novel split recombinant protein provided by the present disclosure was able to restore firefly luciferase activity that provides a notably higher luminescence intensity than that of the conventional firefly luciferase derived from the North American firefly (Photinus pyralis). These Examples also demonstrate that expression of a gene that encodes the split recombinant protein allows gene expression and calcium ion dynamics in cells with weak expression of foreign genes to be analyzed by imaging, and also enables single-cell imaging and high-speed imaging. The gene that encodes the split recombinant protein is similarly suitably applicable to methods of analyzing protein-protein interactions or receptor-receptor interactions.

Further, as seen from the Examples described above, the firefly luciferase activity restored by the split recombinant protein was able to provide different luminescence colors depending on the species of firefly, the number of N-terminal side amino acid residues of the C-terminal fragment, or combinations thereof. This means that the split recombinant protein and the gene encoding the protein are also suitably applicable to multicolor imaging and other techniques.

1. A split recombinant protein comprising:
an N-terminal fragment of a firefly luciferase, the N-terminal fragment being one of two fragments of the firefly luciferase split into two such that activity of the firefly luciferase is restored when the two fragments are bound together;
a C-terminal fragment of a firefly luciferase, the C-terminal fragment including 58 to 78 amino acid residues toward an N-terminal beyond a splitting position at which the firefly luciferase can be split into two such that activity of the firefly luciderase is restored when the two fragments are hound together; and
a linker polypeptide,
wherein firefly luciferase activity is exhibited when the N-terminal and C-terminal fragments are bound together.

2. The split recombinant protein according to claim 1, wherein the N-terminal and C-terminal fragments are each derived from a different firefly luciferase of a different firefly species.

3. The split recombinant protein according to claim 1, wherein the N-terminal and C-terminal fragments are each derived from a firefly luciferase of a firefly selected from the group consisting of Pyrocoelia matsumurai, Drilaster Kumejimensis, and Stenocladius flavipennis.

4. The split recombinant protein according to claim 3, wherein the N-terminal fragment is derived from a firefly luciferase of Pyrocoelia matsumurai.

5. The split recombinant protein according to claim 1, wherein the N-terminal fragment is positioned on a C-terminal side in the split recombinant protein, and the C-terminal fragment is positioned on an N-terminal side in the split recombinant protein.

6. The split recombinant protein according to claim 1, further comprising, between the N-terminal and C-terminal fragments, a calcium-binding region and an interaction region that can reversibly bind to or dissociate from the calcium-binding region.

7. The split recombinant protein according to claim 6, wherein the calcium-binding region is derived from calmodulin, and the interaction region is M13 peptide.

8. A gene that encodes the split recombinant protein according to claim 1.

9. A vector comprising:
a promoter sequence; and
the gene according to claim 8 expressibly linked to the promoter sequence.

10. A cell comprising the vector according to claim 9.

11. A method of analyzing intracellular calcium ions, comprising:
preparing a cell containing a vector that contains a promoter sequence and a gene that encodes the split recombinant protein according to claim 6, the gene being expressibly linked to the promoter sequence;
adding a firefly luciferin to the cell from outside the cell;
measuring a luminescence level in the cell over time; and
analyzing changes in a calcium ion concentration in the cell based on changes in the luminescence level measured.

12. The method according to claim 11, wherein the method uses two or more vectors each containing a different gene that encodes the split recombinant protein having a different luminescence color.

13. The method according to claim 11, wherein the method analyzes changes in a calcium ion concentration within a single cell.

14. A method of analyzing intracellular gene expression, comprising:
preparing a cell containing a vector that contains a promoter sequence, a target gene and a gene that encodes the split recombinant protein according to claim 1, the target gene and the gene being expressibly linked to the promoter sequence;
adding a firefly luciferin to the cell from outside the cell;
measuring a luminescence level in the cell; and
analyzing an expression level of the target gene in the cell based on the luminescence level measured.

15. The method according to claim 14, wherein the method uses two or more vectors each containing a different gene that encodes the split recombinant protein having a different luminescence color.

16. A vector set comprising:
A first vector containing a first promoter sequence and a first gene that encodes an N-terminal fragment of a firefly luciferase is expressibly linked to the first promoter sequence, the N-terminal fragment being one of two fragments of the firefly luciferase split into two such that activity of the firefly luciferase is restored when the two fragments are bound together; and
a second vector containing a second promoter sequence and a second gene that encodes a C-terminal fragment of a firefly luciferase is expressibly linked to the second promoter sequence, the C-terminal fragment including 58 to 78 amino acid residues toward an N-terminal beyond a splitting position at which the firefly luciferase can be split into two such that activity of the firefly luciferase is restored when the two fragments are bound together.

17. The vector set according to claim 16, wherein the N-terminal and C-terminal fragments are each derived from a different firefly luciferase of a different firefly species.

18. The vector set according to claim 16, wherein either one of the and second promoter sequences is an inducible promoter sequence.

19. A method of analyzing intracellular gene expression, comprising:
preparing a cell containing the vector set according to claim 18;
adding a firefly luciferin to the cell from outside the cell;
adding to the cell an inducer that stimulates the inducible promoter sequence;
measuring a luminescence level in the cell; and
analyzing an activity of the inducible promoter sequence in the cell based on the luminescence level measured.

20. A cell comprising the vector set according to claim 16.


SEQUENCE LISTING (68 sequences)

SEQ ID NO: 1 - DNA, 1647 bp, Pyrocoelia matsumurai
atggaggacgaccacaagaacatcgtgcacggcccagccccgttctacccactggaggagggaaccgccggagagcagctgcaccgcgccatgaagcgctatgcccaggtgccaggcaccattgccttcaccgatgcccacgtggaagtgaacatcacctacagcgagtacttcgagatggcctgccgcctggccgagacgatgaagcgctacggactgggcctgcagcaccacattgccgtgtgcagcgagaacagcctgcagttcttcatgcccgtgtgcggagccctgttcatcggagtgggagtggcccccaccaacgacatctacaacgagcgcgagctgtacaacagcctgagcatcagccagcccaccatcgtgttctgcagcaagcgcgccctgcagaagatcctgggcgtgcagaagaagctgcccgtgatcgagaagatcgtgatcctggatagccgcgaggattacatgggcaagcagagcatgtacagcttcatcgagagccatctgccagccggcttcaacgagtacgattacgtgcccgataccttcgatcgcgagaccgccaccgccctgatcatgaatagcagcggcagcaccggcctgcccaagggcgtggagctgacccacaagaatgtgtgcgtgcgcttcagccactgccgcgatcccgtgttcggcaaccagatcatccccgataccgccatcctgaccgtgatcccgttccaccacggcttcggcatgttcaccaccctgggctacctgacctgcggcttccgcatcgtgctgatgtaccgcttcgaggaggagctgttcctgcgctccctgcaggattacaagatccagagcgccctgctggtgccgaccctgttctcgttcttcgccaagagcaccctggtggataagtacgatctgagcaacctgcacgagatcgccagcggcggagccccactggccaaggaagtgggagaggccgtggccaagcgcttcaagctgccaggcattcgccagggctatggcctgaccgagaccaccagcgccgtgattattaccccacgcggcgacgataagccaggcgcctgcggaaaggtggccccattcttcagcgccaagattgtggatctggataccagcaagacgctgggcgtgaaccagcgcggagagctgtgcctgaagggccccatgattatgaagggctacgtgaacaaccccgaggccaccaatgccctgatcgataaggatggctggctgcacagcggcgatctggcctactacgataaggacggccacttcttcatcgtggatcgcctgaagtccctgatcaagtaccagggctaccaggtgcccccagccgagctggagagtatcctgctgcagcatcccttcatcttcgatgccggcgtggccggcatcccagatgccgacgccggcgagctgccagccgccgtggtggtgctggaggagggcaagaccatgaccgagcaggaagtgatggattacgtggccggacaagtgaccgcctccaagcgcctgcgcggaggcgtgaagtttgtggatgaggtgccaaagggcctgacgggcaagatcgatagccgcaagatccgcgagatgctgacgatgggcaagaagtccaagctgtag

SEQ ID NO: 2 - protein, 548 aa, Pyrocoelia matsumurai
MEDDHKNIVHGPAPFYPLEEGTAGEQLHRAMKRYAQVPGTIAFTDAHVEVNITYSEYFEMACRLAETMKRYGLGLQHHIAVCSENSLQFFMPVCGALFIGVGVAPTNDIYNERELYNSLSISQPTIVFCSKRALQKILGVQKKLPVIEKIVILDSREDYMGKQSMYSFIESHLPAGFNEYDYVPDTFDRETATALIMNSSGSTGLPKGVELTHKNVCVRFSHCRDPVFGNQIIPDTAILTVIPFHHGFGMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAVIITPRGDDKPGACGKVAPFFSAKIVDLDTSKTLGVNQRGELCLKGPMIMKGYVNNPEATNALIDKDGWLHSGDLAYYDKDGHFFIVDRLKSLIKYQGYQVPPAELESILLQHPFIFDAGVAGIPDADAGELPAAVVVLEEGKTMTEQEVMDYVAGQVTASKRLRGGVKFVDEVPKGLTGKIDSRKIREMLTMGKKSKL

SEQ ID NO: 3 - protein, 416 aa, Pyrocoelia matsumurai
MEDDHKNIVHGPAPFYPLEEGTAGEQLHRAMKRYAQVPGTIAFTDAHVEVNITYSEYFEMACRLAETMKRYGLGLQHHIAVCSENSLQFFMPVCGALFIGVGVAPTNDIYNERELYNSLSISQPTIVFCSKRALQKILGVQKKLPVIEKIVILDSREDYMGKQSMYSFIESHLPAGFNEYDYVPDTFDRETATALIMNSSGSTGLPKGVELTHKNVCVRFSHCRDPVFGNQIIPDTAILTVIPFHHGFGMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAVIITPRGDDKPGACGKVAPFFSAKIVDLDTSKTLGVNQRGELCLKGPMIMKGYVNNPEATNALIDKD

SEQ ID NO: 4 - protein, 132 aa, Pyrocoelia matsumurai
GWLHSGDLAYYDKDGHFFIVDRLKSLIKYQGYQVPPAELESILLQHPFIFDAGVAGIPDADAGELPAAVVVLEEGKTMTEQEVMDYVAGQVTASKRLRGGVKFVDEVPKGLTGKIDSRKIREMLTMGKKSKL

SEQ ID NO: 5 - DNA, 1644 bp, Drilaster Kumejimensis
atggacatggaagataagaacgtggtgcacggccctgccagcttctaccctgtggatgatggcacagccggccagcagctgcacaagaccatcctgagatacgcccagctgcccggcacaatcgcctttaccgatgcccacaccgagcagaacctgacctacgccgagtacttcgacctgacctgcagactggccgagagcatgaagagatacggcctgggcctgaagcacgccattgccgtgtgcagcgagaacagcctgaacttcttcgcccccgtgatcgccgccctgtatctgggagtggccgtggcccccaccaacgacatctacaacgagagagagctgtacaactccctgaacatcagccagcccaccatcgtgttctgcagcaagcgggccctgctgaagatcatggaagtgcagaaaaagctgcccgtgatccagaaaatcatcgtgctggaccacaaagaggacttccagggcctgcacagcatgcacagcttcatcaagcactacctgcccagccacttcaacgagcacgacttcaagcccgaagagttcgaccggaacgaccaggtggccctgatcatgaacagcagcggcagcaccggcctgcctaaaggcgtgatgctgacccacaagaatgtggtcgtgcggttcagccactgcaaggaccccgtgttcggcaaccagatcatccccgacaccgctatcctgaccgtgatccctttccaccacggcttcggcatgttcaccaccctgggctacctgacctgtggcttccggatcgtggtcatgcacaagttcgaggaagaactgttcctgcggagcctgcaggactacaagatccagagcgccctgctggtgcctaccctgttcagcttcttcgccaagagcaccctggtggataagtacgacctgagcaacctgaaagagatcgccagcggcggagcccccctggctaaagaagtgggagaggccgtcgccaagcggtttaagctgcctggcgtgcggcagggctacggactgacagagacaaccagcgccgtgatcatcacacccgagggcgaagataaggccggctctacaggcaaggtggtgccattcttcagcgccaagatcatcgacctggacaccagacagagcctgggccctcaccagaaaggcgagctgtgcctgaagggcgacatgatcatgaagggctacgccaacaaccccgaggccaccaacgccctgatcgacaaggatggctggctgcacagcggcgacctggcctactacgacgaggacggccacttcttcatcgtggaccggctgaagtccctgatcaagtacaagggctatcaggtggcccctgccgagctggaaagcgtgctgctgcagcatccctacatcttcgacgctggcgtgaccggcatccctgatgaagtggcaggcgaactgcctgccgcctgtgtggtgctggaaaagggcaagcacctgaccgagaaagaagtgatggactacgtcgccagccaggtgtccagcagcaagaaactgagaggcggcgtgcgcttcatcgacgagatccctaagggcctgaccggcaagatcgacagcagaggcctgaaagaaatcctgaagcggccccagagcaagatgtga

SEQ ID NO: 6 - protein, 547 aa, Drilaster Kumejimensis
MDMEDKNVVHGPASFYPVDDGTAGQQLHKTILRYAQLPGTIAFTDAHTEQNLTYAEYFDLTCRLAESMKRYGLGLKHAIAVCSENSLNFFAPVIAALYLGVAVAPTNDIYNERELYNSLNISQPTIVFCSKRALLKIMEVQKKLPVIQKIIVLDHKEDFQGLHSMHSFIKHYLPSHFNEHDFKPEEFDRNDQVALIMNSSGSTGLPKGVMLTHKNVVVRFSHCKDPVFGNQIIPDTAILTVIPFHHGFGMFTTLGYLTCGFRIVVMHKFEEELFLRSLQDYKIQSALLVPTLFSFFAKSTLVDKYDLSNLKEIASGGAPLAKEVGEAVAKRFKLPGVRQGYGLTETTSAVIITPEGEDKAGSTGKVVPFFSAKIIDLDTRQSLGPHQKGELCLKGDMIMKGYANNPEATNALIDKDGWLHSGDLAYYDEDGHFFIVDRLKSLIKYKGYQVAPAELESVLLQHPYIFDAGVTGIPDEVAGELPAACVVLEKGKHLTEKEVMDYVASQVSSSKKLRGGVRFIDEIPKGLTGKIDSRGLKEILKRPQSKM

SEQ ID NO: 7 - protein, 416 aa, Drilaster Kumejimensis
MDMEDKNVVHGPASFYPVDDGTAGQQLHKTILRYAQLPGTIAFTDAHTEQNLTYAEYFDLTCRLAESMKRYGLGLKHAIAVCSENSLNFFAPVIAALYLGVAVAPTNDIYNERELYNSLNISQPTIVFCSKRALLKIMEVQKKLPVIQKIIVLDHKEDFQGLHSMHSFIKHYLPSHFNEHDFKPEEFDRNDQVALIMNSSGSTGLPKGVMLTHKNVVVRFSHCKDPVFGNQIIPDTAILTVIPFHHGFGMFTTLGYLTCGFRIVVMHKFEEELFLRSLQDYKIQSALLVPTLFSFFAKSTLVDKYDLSNLKEIASGGAPLAKEVGEAVAKRFKLPGVRQGYGLTETTSAVIITPEGEDKAGSTGKVVPFFSAKIIDLDTRQSLGPHQKGELCLKGDMIMKGYANNPEATNALIDKD

SEQ ID NO: 8 - protein, 131 aa, Drilaster Kumejimensis
GWLHSGDLAYYDEDGHFFIVDRLKSLIKYKGYQVAPAELESVLLQHPYIFDAGVTGIPDEVAGELPAACVVLEKGKHLTEKEVMDYVASQVSSSKKLRGGVRFIDEIPKGLTGKIDSRGLKEILKRPQSKM

SEQ ID NO: 9 - DNA, 1668 bp, Stenocladius flavipennis
atggccagcagcatgatgagcaagaaggacctggaagataagaacgtggtgcacggccccgacccctactacctggtggatgagggcaatgccggccagcagctgcacaagaccatcctgagatacgcccagctgcccgacacaatcgccttcaccgacggccacaccaagcgggatgtgacctacgcccactacttcgacctgacctgcagactggccgagagcctgaagagatacggcctgaacctgcagagccggatcgccgtgtgcagcgagaacaacgtggaatttttcatccccgtggtggccagcctgtacctgggagtgggagtggcccccaccaacgacatctacaacgagacagagctgttcaacagcctgaacatcagccagcccaccatcgtgttcgtgtccaagcgggccctgcacaagatcctggaagtgaagaagcgcatccccatcatcaagaccgtggtggtgctggacaccgaagaggacttcatgggctaccactgcctgcacagctttatgaagcactacctgccccccaacttcgacatcatgagctacaagcccgaagagttcgcccgggatggacagctggccctgatcatgaacagcagcggcagcaccggcctgcctaaaggcgtgatgctggcccacagatccgtggtcgtgcggttcagccactgcaaggaccccgtgttcggcaaccagatcatccccgacaccgctatcctgaccgtgatccctttccaccacggcttcggcatgttcaccaccctgggctacctgacctgtggcttccggatcgtgctgctgcggaagttcgacgagcactactttctgaagtgcctgcaggactacaagatccagtttgccctgctggtgcctaccctgttcagcttcttcgccaagagcaccctggtggaccagtacgacctgagcaacctgaaagagatcgccagcggcggagcccccctggctaaagaagtgggagaggccgtcgccaagcggtttaagctgcccggcatcagacagggctacggcctgaccgagacaaccagcgccgtgatcatcacccccgagggcgaggataagcctggctctacaggcaaggtggtgccattcttcagcgccaagatcgtggacctgaacagcggcaagagcgtgggccctcaccagaggggagaactctacctgaagggcgacatgatcatgatgggctactgcaacaacaaggccgccaccgacgagatgatcgacaaggatggctggctgcactccggcgacgttgcctactacgacgaggacggccacttcttcatcgtggaccggctgaagtccctgatcaagtacaagggctaccaggtggcccctgccgaactggaagctgtgctgctgcagcatccctgcatcttcgatgccggcgtgaccggcgtgccagatgatgtggacggcgaactgcctggcgcctgtgtggtcctggaaaagggcaagcacgtgaccgagcaggaagtgatggactacgtcgccggccagctgagctgctacaagagactgagaggcggtgtgcgcttcatcgatgagatccctaagggcctgaccggcaagatcgaccggaaggccctgaaagaaatcctgaagaaaccccagagcaagatgtga

SEQ ID NO: 10 - protein, 555 aa, Stenocladius flavipennis
MASSMMSKKDLEDKNVVHGPDPYYLVDEGNAGQQLHKTILRYAQLPDTIAFTDGHTKRDVTYAHYFDLTCRLAESLKRYGLNLQSRIAVCSENNVEFFIPVVASLYLGVGVAPTNDIYNETELFNSLNISQPTIVFVSKRALHKILEVKKRIPIIKTVVVLDTEEDFMGYHCLHSFMKHYLPPNFDIMSYKPEEFARDGQLALIMNSSGSTGLPKGVMLAHRSVVVRFSHCKDPVFGNQIIPDTAILTVIPFHHGFGMFTTLGYLTCGFRIVLLRKFDEHYFLKCLQDYKIQFALLVPTLFSFFAKSTLVDQYDLSNLKEIASGGAPLAKEVGEAVAKRFKLPGIRQGYGLTETTSAVIITPEGEDKPGSTGKVVPFFSAKIVDLNSGKSVGPHQRGELYLKGDMIMMGYCNNKAATDEMIDKDGWLHSGDVAYYDEDGHFFIVDRLKSLIKYKGYQVAPAELEAVLLQHPCIFDAGVTGVPDDVDGELPGACVVLEKGKHVTEQEVMDYVAGQLSCYKRLRGGVRFIDEIPKGLTGKIDRKALKEILKKPQSKM

SEQ ID NO: 11 - protein, 424 aa, Stenocladius flavipennis
MASSMMSKKDLEDKNVVHGPDPYYLVDEGNAGQQLHKTILRYAQLPDTIAFTDGHTKRDVTYAHYFDLTCRLAESLKRYGLNLQSRIAVCSENNVEFFIPVVASLYLGVGVAPTNDIYNETELFNSLNISQPTIVFVSKRALHKILEVKKRIPIIKTVVVLDTEEDFMGYHCLHSFMKHYLPPNFDIMSYKPEEFARDGQLALIMNSSGSTGLPKGVMLAHRSVVVRFSHCKDPVFGNQIIPDTAILTVIPFHHGFGMFTTLGYLTCGFRIVLLRKFDEHYFLKCLQDYKIQFALLVPTLFSFFAKSTLVDQYDLSNLKEIASGGAPLAKEVGEAVAKRFKLPGIRQGYGLTETTSAVIITPEGEDKPGSTGKVVPFFSAKIVDLNSGKSVGPHQRGELYLKGDMIMMGYCNNKAATDEMIDKD

SEQ ID NO: 12 - protein, 131 aa, Stenocladius flavipennis
GWLHSGDVAYYDEDGHFFIVDRLKSLIKYKGYQVAPAELEAVLLQHPCIFDAGVTGVPDDVDGELPGACVVLEKGKHVTEQEVMDYVAGQLSCYKRLRGGVRFIDEIPKGLTGKIDRKALKEILKKPQSKM

SEQ ID NO: 13 - DNA, 30 bp, Artificial Sequence
  OKI-N_Bgl_Fw
agatctgaggacgaccacaagaacatcgtg

SEQ ID NO: 14 - DNA, 30 bp, Artificial Sequence
  OKI-N_Eco_Rv
gaattcatccttatcgatcagggcattggt

SEQ ID NO: 15 - DNA, 36 bp, Artificial Sequence
  OKI399_Bam_Fw
ggatccgccaccatgaagggctacgtgaacaacccc

SEQ ID NO: 16 - DNA, 36 bp, Artificial Sequence
  OKI394_Bam_Fw
ggatccgccaccatgaagggccccatgattatgaag

SEQ ID NO: 17 - DNA, 36 bp, Artificial Sequence
  OKI389_Bam_Fw
ggatccgccaccatgggagagctgtgcctgaagggc

SEQ ID NO: 18 - DNA, 36 bp, Artificial Sequence
  OKI384_Bam_Fw
ggatccgccaccatgggcgtgaaccagcgcggagag

SEQ ID NO: 19 - DNA, 36 bp, Artificial Sequence
  OKI379_Bam_Fw
ggatccgccaccatgaccagcaagacgctgggcgtg

SEQ ID NO: 20 - DNA, 36 bp, Artificial Sequence
  OKI374_Bam_Fw
ggatccgccaccatgattgtggatctggataccagc

SEQ ID NO: 21 - DNA, 36 bp, Artificial Sequence
  OKI369_Bam_Fw
ggatccgccaccatgttcttcagcgccaagattgtg

SEQ ID NO: 22 - DNA, 36 bp, Artificial Sequence
  OKI364_Bam_Fw
ggatccgccaccatgggaaaggtggccccattcttc

SEQ ID NO: 23 - DNA, 36 bp, Artificial Sequence
  OKI359_Bam_Fw
ggatccgccaccatgaagccaggcgcctgcggaaag

SEQ ID NO: 24 - DNA, 39 bp, Artificial Sequence
  OKI354_Bam_Fw
ggatccgccaccatgccacgcggcgacgataagccaggc

SEQ ID NO: 25 - DNA, 39 bp, Artificial Sequence
  OKI349_Bam_Fw
ggatccgccaccatggccgtgattattaccccacgcggc

SEQ ID NO: 26 - DNA, 39 bp, Artificial Sequence
  OKI344_Bam_Fw
ggatccgccaccatgaccgagaccaccagcgccgtgatt

SEQ ID NO: 27 - DNA, 39 bp, Artificial Sequence
  OKI339_Bam_Fw
ggatccgccaccatgcagggctatggcctgaccgagacc

SEQ ID NO: 28 - DNA, 30 bp, Artificial Sequence
  OKI-C_Xho_Rv
ctcgagcagcttggacttcttgcccatcgt

SEQ ID NO: 29 - DNA, 30 bp, Artificial Sequence
  KUME-N_Bgl_Fw
agatctgacatggaagataagaacgtggtg

SEQ ID NO: 30 - DNA, 30 bp, Artificial Sequence
  KUME-N_Eco_Rv
gaattcatccttgtcgatcagggcgttggt

SEQ ID NO: 31 - DNA, 36 bp, Artificial Sequence
  KUME399_Bam_Fw
ggatccgccaccatgaagggctacgccaacaacccc

SEQ ID NO: 32 - DNA, 36 bp, Artificial Sequence
  KUME394_Bam_Fw
ggatccgccaccatgaagggcgacatgatcatgaag

SEQ ID NO: 33 - DNA, 36 bp, Artificial Sequence
  KUME389_Bam_Fw
ggatccgccaccatgggcgagctgtgcctgaagggc

SEQ ID NO: 34 - DNA, 36 bp, Artificial Sequence
  KUME384_Bam_Fw
ggatccgccaccatgggccctcaccagaaaggcgag

SEQ ID NO: 35 - DNA, 36 bp, Artificial Sequence
  KUME379_Bam_Fw
ggatccgccaccatgaccagacagagcctgggccct

SEQ ID NO: 36 - DNA, 36 bp, Artificial Sequence
  KUME374_Bam_Fw
ggatccgccaccatgatcatcgacctggacaccaga

SEQ ID NO: 37 - DNA, 36 bp, Artificial Sequence
  KUME369_Bam_Fw
ggatccgccaccatgttcttcagcgccaagatcatc

SEQ ID NO: 38 - DNA, 36 bp, Artificial Sequence
  KUME364_Bam_Fw
ggatccgccaccatgggcaaggtggtgccattcttc

SEQ ID NO: 39 - DNA, 36 bp, Artificial Sequence
  KUME359_Bam_Fw
ggatccgccaccatgaaggccggctctacaggcaag

SEQ ID NO: 40 - DNA, 39 bp, Artificial Sequence
  KUME354_Bam_Fw
ggatccgccaccatgcccgagggcgaagataaggccggc

SEQ ID NO: 41 - DNA, 39 bp, Artificial Sequence
  KUME349_Bam_Fw
ggatccgccaccatggccgtgatcatcacacccgagggc

SEQ ID NO: 42 - DNA, 39 bp, Artificial Sequence
  KUME344_Bam_Fw
ggatccgccaccatgacagagacaaccagcgccgtgatc

SEQ ID NO: 43 - DNA, 39 bp, Artificial Sequence
  KUME339_Bam_Fw
ggatccgccaccatgcagggctacggactgacagagaca

SEQ ID NO: 44 - DNA, 30 bp, Artificial Sequence
  KUME-C_Xho_Rv
ctcgagcatcttgctctggggccgcttcag

SEQ ID NO: 45 - DNA, 30 bp, Artificial Sequence
  SfRE-N_Bgl_Fw
agatctgccagcagcatgatgagcaagaag

SEQ ID NO: 46 - DNA, 30 bp, Artificial Sequence
  SfRE-N_Eco_Rv
gaattcatccttgtcgatcatctcgtcggt

SEQ ID NO: 47 - DNA, 36 bp, Artificial Sequence
  SfRE407_Bam_Fw
ggatccgccaccatgatgggctactgcaacaacaag

SEQ ID NO: 48 - DNA, 36 bp, Artificial Sequence
  SfRE402_Bam_Fw
ggatccgccaccatgaagggcgacatgatcatgatg

SEQ ID NO: 49 - DNA, 36 bp, Artificial Sequence
  SfRE397_Bam_Fw
ggatccgccaccatgggagaactctacctgaagggc

SEQ ID NO: 50 - DNA, 36 bp, Artificial Sequence
  SfRE392_Bam_Fw
ggatccgccaccatgggccctcaccagaggggagaa

SEQ ID NO: 51 - DNA, 36 bp, Artificial Sequence
  SfRE387_Bam_Fw
ggatccgccaccatgagcggcaagagcgtgggccct

SEQ ID NO: 52 - DNA, 36 bp, Artificial Sequence
  SfRE382_Bam_Fw
ggatccgccaccatgatcgtggacctgaacagcggc

SEQ ID NO: 53 - DNA, 36 bp, Artificial Sequence
  SfRE377_Bam_Fw
ggatccgccaccatgttcttcagcgccaagatcgtg

SEQ ID NO: 54 - DNA, 36 bp, Artificial Sequence; identical to SEQ ID NO: 38
  SfRE372_Bam_Fw

SEQ ID NO: 55 - DNA, 36 bp, Artificial Sequence
  SfRE367_Bam_Fw
ggatccgccaccatgaagcctggctctacaggcaag

SEQ ID NO: 56 - DNA, 39 bp, Artificial Sequence
  SfRE362_Bam_Fw
ggatccgccaccatgcccgagggcgaggataagcctggc

SEQ ID NO: 57 - DNA, 39 bp, Artificial Sequence
  SfRE357_Bam_Fw
ggatccgccaccatggccgtgatcatcacccccgagggc

SEQ ID NO: 58 - DNA, 39 bp, Artificial Sequence
  SfRE352_Bam_Fw
ggatccgccaccatgaccgagacaaccagcgccgtgatc

SEQ ID NO: 59 - DNA, 39 bp, Artificial Sequence
  SfRE347_Bam_Fw
ggatccgccaccatgcagggctacggcctgaccgagaca

SEQ ID NO: 60 - DNA, 30 bp, Artificial Sequence
  SfRE-C_Xho_Rv
ctcgagcatcttgctctggggtttcttcag

SEQ ID NO: 61 - DNA, 28 bp, Artificial Sequence
  C-fos_pro_Fw
agctcgagagcagttcccgtcaatccct

SEQ ID NO: 62 - DNA, 27 bp, Artificial Sequence
  C-fos_pro_Rv
caaagctttgcagaagtcctagaacaa

SEQ ID NO: 63 - DNA, 30 bp, Artificial Sequence
  SYN_pro_Fw
ctcgaggccacattggcactggatgtttcc

SEQ ID NO: 64 - DNA, 30 bp, Artificial Sequence
  SYN_pro_Rv
aagcttgacttggggcagggggtcctaggg

SEQ ID NO: 65 - DNA, 30 bp, Artificial Sequence
  CaM-M13_Fw
gccctcgagcatgaccaactgacagaagag

SEQ ID NO: 66 - DNA, 30 bp, Artificial Sequence
  CaM-M13_Rv
catggatcccagtgccccggagctggagat

SEQ ID NO: 67 - DNA, 30 bp, Artificial Sequence
  HU_5HT2AR_Fw
gccaccatggatattctttgtgaagaaaat

SEQ ID NO: 68 - DNA, 30 bp, Artificial Sequence
  HU_5HT2AR_Rv
tcacacacagctcaccttttcattcactcc